(19) United States
(12) Patent Application Publication
(10) Pub. No.: US 2026/0256874 A1
(43) Pub. Date: Sep. 3, 2026

(54) PHARMACEUTICAL FORMULATIONS FOR LONGEVITY IN MAMMALS

(71) Applicant: Cellular Longevity, Inc., San Francisco, CA (US)

(72) Inventors: Ariel Howard Earle COHEN, San Francisco, CA (US); Laura VASQUEZ-BOLANOS, San Francisco, CA (US); Celine-Lea HALIOUA-HAUBOLD, San Francisco, CA (US); Michael LACROIX-FRALISH, San Francisco, CA (US); Dina JUAREZ-SALINAS, San Francisco, CA (US); Karen GREENWOOD, San Francisco, CA (US); Aiva SIMAITE, San Francisco, CA (US); Duong NGUYEN, San Francisco, CA (US); Ivan TERZIC, San Francisco, CA (US); Rob STEENDAM, San Francisco, CA (US)

(21) Appl. No.: 19/469,907

(22) PCT Filed: Mar. 27, 2024
(86) PCT No.: PCT/US2024/021723

§ 371 Date: Sep. 26, 2025

Related U.S. Application Data
(60) Provisional application No. 63/492,464, filed on Mar. 27, 2023.

(51) Int. Cl. A61K 38/12; A61K 9/16

(52) U.S. Cl. CPC A61K 38/12; A61K 9/1647

(57) ABSTRACT

Disclosed herein are extended release octreotide dosage forms containing octreotide, or a pharmaceutically acceptable salt thereof, and a biodegradable polymer microsphere. Disclosed herein are also kits of formulation including a first vial containing a microsphere loaded with octreotide, or a pharmaceutically acceptable salt thereof, and a biodegradable polymer and a second vial including a pharmaceutically acceptable diluent. Also, disclosed herein are methods of preventing or reducing an age-related decline in quality of life or an age-related increase in frailty in a mammal by administering to the mammal a therapeutically effective amount of the extended release octreotide dosage form or providing the kit of formulation to the mammal.

### CROSS REFERENCE TO RELATED APPLICATIONS

This application claims the benefit of U.S. Provisional Application Ser. No. 63/492,464, filed Mar. 27, 2023, which is hereby incorporated by reference in its entirety.

### BACKGROUND

Chronological age is well understood to be the single greatest risk factor for nearly every major cause of mortality and morbidity in living organisms, including humans and companion dogs. Even before the development of observable disease, the physiology of organ systems and tissues progressively declines throughout life. Throughout history, products and methods that promote longevity and extend lifespan have been eagerly sought. Generally, these products and methods have proven to be ineffective and/or unsafe. Accordingly, there remains an unmet need for safe and effective products and methods that promote longevity and extend lifespan.

### SUMMARY

The present disclosure addresses this need. Accordingly, in one aspect, the present application discloses extended release octreotide dosage forms, kits of formulation, and methods for increasing lifespan, promoting longevity, and/or preventing, reducing the severity of, or delaying the onset of various aging-associated conditions in a mammal. The extended release octreotide dosage forms, kits of formulation, and methods further improve the lifespan of the mammal.

In one aspect, disclosed herein is an extended release octreotide dosage form comprising: (a) octreotide, or a pharmaceutically acceptable salt thereof, and (b) a biodegradable polymer microsphere, wherein the biodegradable polymer microsphere comprises about 10 wt % to 99 wt % of a biodegradable polymer, wherein the biodegradable polymer is a multiblock copolymer comprising at least one hydrolysable pre-polymer (A) segment and at least one hydrolysable pre-polymer (B) segment; wherein the segments are linked by a multifunctional chain extender; and wherein the octreotide, or the pharmaceutically acceptable salt thereof, is present in the microsphere. In some embodiments, the dosage form comprises about 5 wt % to about 60 wt % of the biodegradable polymer microsphere. In some embodiments, the dosage form comprises about 5 wt % to about 50 wt % of the biodegradable polymer microsphere. In some embodiments, the dosage form comprises about 5 wt % to about 20 wt % of the biodegradable polymer microsphere. In some embodiments, the dosage form comprises about 10 wt % to about 20 wt % of the biodegradable polymer microsphere. In some embodiments, the microsphere comprises about 30 wt % to about 95 wt % of the biodegradable polymer. In some embodiments, the microsphere comprises about 65 wt % to about 95 wt % of the biodegradable polymer. In some embodiments, the biodegradable polymer is a biodegradable multiblock copolymer comprising one hydrolysable pre-polymer (A) segment and one hydrolysable pre-polymer (B) segment. In some embodiments, the segments are randomly distributed over the chain of the biodegradable multiblock copolymer. In some embodiments, the amount of the at least one hydrolysable pre-polymer (A) segment in the biodegradable multiblock copolymer is about 5% to about 50% based on total weight of the biodegradable multiblock copolymer. In some embodiments, the amount of the at least one hydrolysable pre-polymer (A) segment in the biodegradable multiblock copolymer is about 10% based on total weight of the biodegradable multiblock copolymer. In some embodiments, the at least one hydrolysable pre-polymer (A) segment comprises poly(ethylene glycol). In some embodiments, the at least one hydrolysable pre-polymer (A) segment comprises a reaction product of initiators selected from diols, dicarboxylic acids, and hydroxycarboxylic acids. In some embodiments, the at least one hydrolysable pre-polymer (A) segment comprises a reaction product of ester forming monomers selected from glycolide, lactide (D and/or L), ε-caprolactone, and δ-valerolactone. In some embodiments, the at least one hydrolysable pre-polymer (A) segment comprises reaction products of poly(ethylene glycol) and D,L-lactide. In some embodiments, the at least one hydrolysable pre-polymer (A) segment is poly(D,L-lactide)-co-poly(ethylene glycol)-co-poly(D,L-lactide). In some embodiments, the at least one hydrolysable pre-polymer (A) segment has a Mₙ of about 500 g/mol or more, about 700 g/mol or more, about 1000 g/mol or more, 1200 g/mol or more, about 2000 g/mol or more, about 3000 g/mol or more, or about 4000 g/mol or more. In some embodiments, the at least one hydrolysable pre-polymer (A) segment has a Mₙ of about 2000 g/mol or more. In some embodiments, the at least one hydrolysable pre-polymer (B) segment comprises reaction products of diols, L-lactide, and glycolide. In some embodiments, the amount of the at least one hydrolysable pre-polymer (B) segment in the biodegradable multiblock copolymer is about 50% to about 95% based on total weight of the biodegradable multiblock copolymer. In some embodiments, the amount of the at least one hydrolysable pre-polymer (B) segment in the biodegradable multiblock copolymer is about 90% based on total weight of the biodegradable multiblock copolymer. In some embodiments, the at least one hydrolysable pre-polymer (B) segment comprises poly(L-lactide-co-glycolide). In some embodiments, the at least one hydrolysable pre-polymer (B) segment comprises poly(L-lactide-co-glycolide) with a Mₙ of about 1000 g/mol or more, about 1200 g/mol or more, about 2000 g/mol or more, about 3000 g/mol or more, or about 4000 g/mol or more. In some embodiments, the at least one hydrolysable pre-polymer (B) segment comprises poly(L-lactide-co-glycolide) with a glycolide/L-lactide monomer molar ratio of about 0.01 or more, about 0.03 or more, about 0.05 or more, about 0.1 or more, about 0.2 or more, about 0.3 or more, about 0.4 or more, about 0.5 or more, or about 1.0 or more. In some embodiments, the multifunctional chain extender is a difunctional aliphatic chain extender. In some embodiments, the poly(ethylene glycol) has a Mₙ of about 150 to about 5000 g/mol, about 200 g/mol to about 1500 g/mol, about 600 to about 1000 g/mol, about 400 to about 3000 g/mol, about 600 to about 1500 g/mol, about 600 to about 5000 g/mol, or about 1000 to about 3000 g/mol. In some embodiments, the biodegradable polymer microsphere has a swelling ratio under physiological conditions of about 1 to about 4, about 1 to about 2, or about 1 to about 1.5. In some embodiments, the biodegradable multiblock copolymer comprises a [poly(D,L-Lactide)-co-poly(ethylene glycol)-co-poly(D,L-Lactide)]-b-[poly(L-lactide-co-glycolide)] multiblock copolymer.

In one aspect, disclosed herein is an extended release octreotide dosage form comprising: (a) octreotide, or a pharmaceutically acceptable salt thereof, (b) a biodegradable polymer microsphere; and (c) a diluent or injection vehicle; wherein the octreotide or the pharmaceutically acceptable salt thereof, is present in the biodegradable polymer microsphere, wherein the biodegradable polymer microsphere comprises about 10 wt % to 99 wt % of a biodegradable polymer, wherein the biodegradable polyene is a multiblock copolymer, wherein the biodegradable multiblock copolymer comprises a [poly(D,L-Lactide)-co-poly(ethylene glycol)-co-poly(D,L-Lactide)]-b-[poly(L-lactide-co-glycolide)] multiblock copolymer; and wherein the biodegradable multiblock copolymer is represented by [(R¹ₘ R²ₙ R³ₘ)ₚ]ᵣ[(R⁴)_{q})]ₛ, wherein: R¹ and R³ are each a poly(D,L-Lactide) segment; R² is a poly(ethylene glycol) segment; R⁴ is a poly(L-lactide-co-glycolide) segment, wherein the L-lactide:glycolide molar ratio is 99:1 to 80:20 mole/mole; m is a molecular weight of R¹ and R³, and m is 400 to 1000 g/mole; n is a molecular weight of R², and n is 100 to 1200 g/mole; p is a molecular weight of the (R¹ₘ R²ₙ R³ₘ) segment, and p is 1800 to 2200 g/mole; q is a molecular weight of the (R⁴) segment, and q is 3800 to 4200 g/mole; and r/s is a ratio of the (R¹ₘ R²ₙ R³ₘ) segment over the (R⁴), and r/s is 5/95 to 15/85 molar ratio.

The biodegradable multiblock copolymer may be represented by

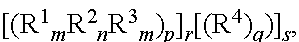

wherein R¹ and R³ are each a poly(DL-Lactide) segment; R² is a poly(ethylene glycol) segment; R⁴ is a poly(L-lactide-co-glycolide) segment;
m is the molecular weight of R¹ and R³;
n is the molecular weight of R²;
p is the molecular weight of the (R¹ₘ R²ₙ R³ₘ) segment;
q is the molecular weight of the (R⁴) segment;
r/s is the molar ratio of the (R¹ₘ R²ₙ R³ₘ) segment over the (R⁴) segment.

In some embodiments, the biodegradable multiblock copolymer as represented by [(R¹ₘ R²ₙ R³ₘ)ₚ]ᵣ[(R⁴)_{q})]ₛ is a [poly(D,L-Lactide)-co-poly(ethylene glycol)-co-poly(D,L-Lactide)]-b-[poly(L-lactide-co-glycolide)] multiblock copolymer wherein the L-lactide:glycolide molar ratio of R⁴ is 90:10 to 80:20 mole/mole, m is 400-600 g/mole, n is 800-1200 g/mole, p is 1800-2200 g/mole, q is 3800-4200 g/mole, and r/s is 5/95-15/85 molar ratio. In some embodiments, the biodegradable multiblock copolymer as represented by [(R¹ₘ R²ₙ R³ₘ)ₚ]ᵣ[(R⁴)_{q})]ₛ is a [poly(D,L-Lactide)-co-poly(ethylene glycol)-co-poly(D,L-Lactide)]-b-[poly(L-lactide-co-glycolide)] multiblock copolymer wherein the L-lactide:glycolide molar ratio of R⁴ is 99:1 to 95:5 mole/mole, m is 800-1000 g/mole, n is 100-300 g/mole, p is 1800-2200 g/mole, q is 3800-4200 g/mole, and r/s is 5/95-15/85 molar ratio. In some embodiments, the biodegradable multiblock copolymer as represented by [(R¹ₘ R² R³ₘ)ₚ]ᵣ[(R⁴)_{q})]ₛ is a [poly(D,L-Lactide)-co-poly(ethylene glycol)-co-poly(D,L-Lactide)]-b-[poly(L-lactide-co-glycolide)] multiblock copolymer wherein the L-lactide:glycolide molar ratio of R⁴ is 95:5 to 90:10 mole/mole, m is 600-800 g/mole, n is 300-800 g/mole, p is 1800-2200 g/mole, q is 3800-4200 g/mole, and r/s is 5/95-15/85 molar ratio.

In some embodiments, the microsphere comprises about 5 wt % to about 30 wt % of the octreotide, or the pharmaceutically acceptable salt thereof. In some embodiments, the microsphere comprises about 10 wt % to about 20 wt % of the octreotide, or the pharmaceutically acceptable salt thereof. In some embodiments, the microsphere comprises about 10 wt % or about 15 wt % of the octreotide, or the pharmaceutically acceptable salt thereof. In some embodiments, the pharmaceutically acceptable salt of octreotide comprises an acetate salt, trifluoroacetate salt, or HCl salt of octreotide. In some embodiments, the pharmaceutically acceptable salt of octreotide comprises an acetate salt of octreotide. In some embodiments, the dosage form further comprises a diluent. In some embodiments, the diluent is an aqueous diluent. In some embodiments, the microspheres are suspended in the aqueous diluent. In some embodiments, the aqueous diluent comprises sodium carboxymethyl cellulose. In some embodiments, the dosage form further comprises a surfactant, e.g., about 0.025 wt % to about 15 wt % of the surfactant. In some embodiments, the dosage form comprises about 0.05 wt % to about 1.0 wt % of the surfactant. In some embodiments, the dosage form comprises about 1.0 wt % to about 10 wt % of the surfactant. In some embodiments, the dosage form comprises about 5 wt % of the surfactant. In some embodiments, the surfactant is selected from a polysorbates, a poloxamers, bile salts, glyceryl monostearate, sodium lauryl sulfate, sorbitan monooleate, polyoxyethylene sorbitan monooleate, copolymers of ethylene oxide and propylene oxide, and d-α-tocopheryl polyethylene glycol succinate (Vitamin E TPGS). In some embodiments, the surfactant comprises a polysorbate, a poloxamer, or a combination thereof. In some embodiments, the surfactant comprises a polysorbate. In some embodiments, within the first 20 days after being administered to a mammal, the microsphere releases from about 5 wt % to about 50 wt % of the octreotide, or the pharmaceutically acceptable salt thereof, present in the microsphere. In some embodiments, within the first 20 days after being administered to a mammal, the microsphere releases from about 20 wt % to about 50 wt % of the octreotide, or the pharmaceutically acceptable salt thereof, present in the microsphere In some embodiments, within the first 60 days after being administered to a mammal, the microsphere releases from about 30 wt % to about 90 wt % of the octreotide, or the pharmaceutically acceptable salt thereof, present in the microsphere. In some embodiments, within the first 60 days after being administered to a mammal, the microsphere releases from about 70 wt % to about 90 wt % of the octreotide, or the pharmaceutically acceptable salt thereof, present in the microsphere. In some embodiments, within the first 20 days after being administered to a mammal, the microsphere releases at most about 10 wt % of the octreotide, or the pharmaceutically acceptable salt thereof, present in the microsphere. In some embodiments, within the first 20 days after being administered to a mammal, the microsphere releases at most about 5 wt % of the octreotide, or the pharmaceutically acceptable salt thereof, present in the microsphere. In some embodiments, within the first 60 days after being administered to a mammal, the microsphere releases at most about 50 wt % of the octreotide, or the pharmaceutically acceptable salt thereof present in the microsphere. In some embodiments, within the first 60 days after being administered to a mammal the microsphere releases at most about 10 wt % of the octreotide, or the pharmaceutically acceptable salt thereof present in the microsphere. In some embodiments, the release is determined by an in vivo Bicinchoninic acid (BCA) assay. In some embodiments, the release is determined by an in vitro LC/MS method.

In one aspect, disclosed herein is a kit of formulation comprising: (a) a first container (e.g., a vial) comprising a biodegradable polymer microsphere, wherein the biodegradable polymer microsphere comprises octreotide, or a pharmaceutically acceptable salt thereof, and a biodegradable polymer; and (b) a second container comprising a pharmaceutically acceptable diluent. In some embodiments, the biodegradable polymer microsphere comprises about 10 wt % to about 99 wt % of the biodegradable polymer. In some embodiments, the biodegradable polymer is a multiblock copolymer comprising at least one hydrolysable pre-polymer (A) segment and at least one hydrolysable pore-polymer (B) segment. In some embodiments, the segments are linked by a multifunctional chain extender. In some embodiments, the octreotide, or the pharmaceutically acceptable salt thereof, is present in the microsphere. In some embodiments, the biodegradable polymer microsphere comprises about 30 wt % to about 95 wt % of a biodegradable polymer. In some embodiments, the biodegradable polymer microsphere comprises about 65 wt % to about 95 wt % of the biodegradable polymer. In some embodiments, the microsphere comprises about 80 wt % to about 90 wt % of the biodegradable polymer. In some embodiments, the biodegradable polymer is a biodegradable multiblock copolymer comprising one hydrolysable pre-polymer (A) segment and one hydrolysable pre-polymer (B) segment. In some embodiments, the segments are randomly distributed over the chain of the biodegradable multiblock copolymer. In some embodiments, the amount of the at least one hydrolysable pre-polymer (A) segment in the biodegradable multiblock copolymer is about 5% to about 50% based on total weight of the biodegradable multiblock copolymer. In some embodiments, the amount of the at least one hydrolysable pre-polymer (A) segment in the biodegradable multiblock copolymer is about 10% based on total weight of the biodegradable multiblock copolymer. In some embodiments, the at least one hydrolysable pre-polymer (A) segment comprises poly(ethylene glycol). In some embodiments, the at least one hydrolysable pre-polymer (A) segment comprises a reaction product of initiators selected from diols, dicarboxylic acids, and hydroxycarboxylic acids. In some embodiments, the at least one hydrolysable pre-polymer (A) segment comprises a reaction product of ester forming monomers selected from glycolide, lactide (D and/or L), ε-caprolactone, and δ-valerolactone. In some embodiments, the at least one hydrolysable pre-polymer (A) segment comprises a reaction product of poly(ethylene glycol) and D,L-lactide. In some embodiments, the one hydrolysable pre-polymer (A) segment is poly(D,L-lactide)-co-poly(ethylene glycol)-co-poly(D,L-lactide). In some embodiments, the at least one hydrolysable pre-polymer (A) segment has a Mₙ of about 500 g/mol or more, about 700 g/mol or more, about 1000 g/mol or more, 1200 g/mol or more, about 2000 g/mol or more, about 3000 g/mol or more, or about 4000 g/mol or more. In some embodiments, the at least one hydrolysable pre-polymer (A) segment has a Mₙ of about 1200 g/mol or more or about 2000 g/mol or more. In some embodiments, the at least one hydrolysable pre-polymer (B) segment comprises a reaction product of diols, L-lactide, and glycolide. In some embodiments, the amount of the at least one hydrolysable pre-polymer (B) segment in the biodegradable multiblock copolymer is about 50% to about 95% based on total weight of the biodegradable multiblock copolymer. In some embodiments, the amount of the at least one hydrolysable pre-polymer (B) segment in the biodegradable multiblock copolymer is about 90% based on total weight of the biodegradable multiblock copolymer. In some embodiments, the at least one hydrolysable pre-polymer (B) segment comprises poly(L-lactide-co-glycolide) with a Mₙ of about 1000 g/mol or more, about 1200 g/mol or more, about 2000 g/mol or more, about 3000 g/mol or more, or about 4000 g/mol or more. In some embodiments, the one hydrolysable pre-polymer (B) segment comprises poly(L-lactide-co-glycolide) with Mₙ of about 1200 g/mol or more or about 2000 g/mol or more. In some embodiments, the at least one hydrolysable pre-polymer (B) segment comprises poly(L-lactide-co-glycolide) with a glycolide/L-lactide monomer molar ratio of about 0.01 or more, about 0.03 or more, about 0.05 or more, about 0.1 or more, about 0.2 or more, about 0.3 or more, about 0.4 or more, or about 0.5 or more. In some embodiments, the one hydrolysable pore-polymer (B) segment is poly(L-lactide-co-glycolide) with a with a glycolide/L-lactide monomer molar ratio of 3/97. In some embodiments, the multifunctional chain extender is a difunctional aliphatic chain extender. In some embodiments, the poly(ethylene glycol) has a Mₙ of about 150 to about 5000 g/mol, about 200 g/mol to about 1500 g/mol, about 600 to about 1000 g/mol, about 400 to about 3000 g/mol, about 600 to about 1500 g/mol, about 600 to about 5000 g/mol, or about 1000 to about 3000 g/mol. In some embodiments, the biodegradable polymer microsphere has a swelling ratio under physiological conditions of about 1 to about 4, about 1 to about 2, or about 1 to about 1.5. In some embodiments, the biodegradable multiblock copolymer comprises a [poly(D,L-Lactide-co-poly(ethylene glycol)-co-poly(D,L-Lactide)]-b-[poly(L-lactide-co-glycolide)] multiblock copolymer. In some embodiments, the biodegradable multiblock copolymer as represented by [(R¹ₘ R² R³ₘ)ₚ]ᵣ[(R⁴)_{q})]ₛ is a [poly(D,L-Lactide)-co-poly(ethylene glycol)-co-poly(D,L-Lactide)]-b-[poly(L-lactide-co-glycolide)] multiblock copolymer wherein the L-lactide:glycolide molar ratio of R⁴ is 90:10 to 80:20 mole/mole, m is 400-600 g/mole, n is 800-1200 g/mole, p is 1800-2200 g/mole, q is 3800-4200 g/mole, and r/s is 5/95-15/85 molar ratio. In some embodiments, the biodegradable multiblock copolymer as represented by [(R¹ₘ R²ₙ R³ₘ)ₚ]ᵣ[(R⁴)_{q})]ₛ is a [poly(D,L-Lactide)-co-poly(ethylene glycol)-co-poly(D,L-Lactide)]-b-[poly(L-lactide-co-glycolide)] multiblock copolymer wherein the L-lactide:glycolide molar ratio of R⁴ is 99:1 to 95:5 mole/mole, m is 800-1000 g/mole, n is 100-300 g/mole, p is 1800-2200 g/mole, q is 3800-4200 g/mole, and r/s is 5/95-15/85 molar ratio. In some embodiments, the biodegradable multiblock copolymer as represented by [(R¹ₘ R²ₙ R³ₘ)ₚ]ᵣ[(R⁴)_{q})]ₛ is a [poly(D,L-Lactide)-co-poly(ethylene glycol)-co-poly(D,L-Lactide)]-b-[poly(L-lactide-co-glycolide)] multiblock copolymer wherein the L-lactide:glycolide molar ratio of R⁴ is 95:5 to 90:10 mole/mole, m is 600-800 g/mole, n is 300-800 g/mole, p is 1800-2200 g/mole, q is 3800-4200 g/mole, and r/s is 5/95-15/85 molar ratio. In some embodiments, the pharmaceutically acceptable salt of octreotide comprises an acetate salt, trifluoroacetate salt, or HCl salt of octreotide. In some embodiments, the pharmaceutically acceptable salt of octreotide comprises an acetate salt of octreotide. In some embodiments, the microsphere is in a powder form. In some embodiments, the pharmaceutically acceptable diluent is an aqueous diluent. In some embodiments, the aqueous diluent comprises carboxymethyl cellulose aqueous solution. In some embodiments, the pharmaceutically acceptable diluent comprises about 0.025 wt % to about 10 wt % of a surfactant. In some embodiments, the pharmaceutically acceptable diluent comprises about 0.05 wt % to about 5 wt % of the surfactant. In some embodiments, the pharmaceutically acceptable diluent comprises about 0.1 wt % to about 2 wt % of the surfactant. In some embodiments, the surfactant is selected from polysorbates, poloxamers, bile salts, glyceryl monostearate, sodium lauryl sulfate, sorbitan monooleate, polyoxyethylene sorbitan monooleate, copolymers of ethylene oxide and propylene oxide, and d-α-tocopheryl polyethylene glycol succinate (Vitamin E TPGS). In some embodiments, the surfactant comprises a polysorbate, poloxamer, or a combination thereof. In some embodiments, the surfactant comprises a polysorbate (e.g., polysorbate 80). In some embodiments, the kit further comprises an instruction for reconstitution.

In one aspect, disclosed herein is a kit of formulation comprising: (a) a first container (e.g., a vial) comprising a biodegradable polymer microsphere, wherein the biodegradable polymer microsphere comprises octreotide, or a pharmaceutically acceptable salt thereof, and a biodegradable polymer, the biodegradable polymer is a multiblock copolymer, wherein the biodegradable multiblock copolymer comprises a [poly(D,L-Lactide-co-poly(ethylene glycol)-co-poly(D,L-Lactide)]-b-[poly(L-lactide-co-glycolide)] multiblock copolymer, wherein the biodegradable multiblock copolymer is represented by [(R¹ₘ R²ₙ R³ₘ)ₚ]ᵣ[(R⁴)_{q})]ₛ, wherein: R¹ and R³ are each a poly(D,L-Lactide) segment; R² is a poly(ethylene glycol) segment; R⁴ is a poly(L-lactide-co-glycolide) segment, wherein the L-lactide:glycolide molar ratio is 99:1 to 80:20 mole/mole; m is a molecular weight of R¹ and R³, and m is 400 to 1000 g/mole; n is a molecular weight of R², and n is 100 to 1200 g/mole; p is a molecular weight of the (R¹ₘ R²ₙ R³ₘ) segment, and p is 1800 to 2200 g/mole; q is a molecular weight of the (R⁴) segment, and q is 3800 to 4200 g/mole; and r/s is a ratio of the (R¹ₘ R²ₙ R³ₘ) segment over the (R⁴), and r/s is 5/95 to 15/85 molar ratio; and (b) a second container comprising a pharmaceutically acceptable diluent.

In another aspect, disclosed herein is a method of making an extended release octreotide dosage form disclosed herein, comprising mixing the first vial and the second vial of a kit disclosed herein.

In another aspect, disclosed herein is a method of preventing or reducing an age-related decline in quality of life in a mammal, comprising administering to the mammal a therapeutically effective amount of the extended release octreotide dosage form disclosed herein, or providing the kit disclosed herein to the mammal.

In another aspect, disclosed herein is a method of preventing or reducing an age-related increase in frailty in a mammal, comprising administering to the mammal a therapeutically effective amount of the extended release octreotide dosage form disclosed herein, or providing the kit disclosed herein to the mammal.

In another aspect, disclosed herein is a method of improving cognitive function in a mammal in need thereof, comprising administering to the mammal a therapeutically effective amount of the extended release octreotide dosage form disclosed herein, or providing the kit disclosed herein to the mammal.

In another aspect, disclosed herein is a method of increasing lifespan, promoting longevity, and/or preventing, reducing the severity of or delaying the onset of various aging-associated conditions in a mammal in need thereof, comprising administering to the mammal a therapeutically effective amount of the extended release octreotide dosage form disclosed herein, or providing the kit disclosed herein to the manual. In some embodiments, increasing lifespan comprises an at least 5% increase in lifespan relative to the expected or median lifespan of a mammal of similar species, strain, or breed. In some embodiments, increasing lifespan comprises an at least 10%, at least 15%, at least 20%, or at least 25% increase in lifespan. In some embodiments, the therapeutically effective amount of the pharmaceutical formulation inhibits production and/or release of growth hormone. In some embodiments, the therapeutically effective amount of the pharmaceutical formulation inhibits growth hormone receptor activity. In some embodiments, the therapeutically effective amount of the pharmaceutical formulation produces a decrease in the amount of activity along the growth hormone-growth hormone receptor axis. In some embodiments, the mammal has reached maturity. In some embodiments, the mammal has reached old age. In some embodiments, the mammal is dog, cat, horse, cow, pig, rabbit, rodent, sheep, non-human primate, or human. In some embodiments, the mammal is a dog. In some embodiments, the rodent is a mouse or a rat. In some embodiments, the mammal is a human.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### BRIEF DESCRIPTION OF THE FIGURES

FIG. 1 is a graphical representation of the association between age and standardized log(insulin) for dogs in different weight groups.

FIG. 2A is a graphical representation of the association between insulin and a health-related quality of life assessment (HRQL).

FIG. 2B is a graphical representation of the association between insulin and Canine Frailty Index (CFI).

FIG. 3A is a graph showing the HRQL scores for each insulin tertile group, adjusting for covariates.

FIG. 3B depicts the HRQL kernel density estimate.

FIG. 3C depicts the HRQL total score on the Davies scale as a function of age.

FIG. 4A is a graph showing the CFI score in relation to age.

FIG. 4B is a graph showing the CFI score in relation to total HRQL score.

FIGS. 5A-5F depict insulin or glucose tolerance tested after treatment with SandostatinLAR: baseline fasted insulin (FIG. 5A); Day 70 fasted insulin (FIG. 5B); change in fasted insulin (FIG. 5C); Day 70 fasted glucose (FIG. 5D and FIG. 5E); and change in fasted glucose (FIG. 5F).

FIGS. 6A-6E depict insulin levels gathered and analyzed after treatment with SandostatinLAR: a mixed model insulin sensitivity (Si) (FIG. 6A); a mixed model acute insulin response (AIRg) (FIG. 6B); a mixed model disposition index (DI) (FIG. 6C); a mixed model glucose tolerance (Kg) (FIG. 6D); and a mixed model glucose effectiveness (Sg) (FIG. 6E). (*=p<0.05; **=p<0.01; ***=p<0.001; ****=p<0.0001).

FIG. 7 shows SEM images of OCT-MSP with 20 wt. % target OCT loading prepared of 10LP10L20-GLL40 (15/85) via W/O/W double emulsification process using 0.4 wt. % PVA/5 wt. % NaCl in UPW (174A-220403) or 0.4 wt. % PVA/5 wt % NaCl in 50 mM TRIS buffer pH 8.3 (174A-220404) as CP.

FIG. 8 depicts cumulative release of OCT from OCT-MSP with 20 wt. % target OCT loading prepared of 10LP10L20-GLL40 (15/85) via W/O/W membrane emulsification using 0.4 wt. % PVA/5 wt. % NaCl in UPW (174A-220403) or 0.4 wt. % PVA/5 wt % NaCl in 50 mM TRIS buffer pH 8.3 (174A-220404) as CP.

FIG. 9 depicts cumulative release of OCT from OCT-MSP with 10 or 15 wt. % OCT target loading prepared of 10LP2L20-GLL40 (3/97) and 10GL20-L40 via W/O/W membrane emulsification.

FIG. 10 depicts cumulative release of OCT from microspheres with 20 wt. % target OCT loading prepared of 10LP2L20-GLL40 (3/97) via O/W membrane emulsification procedure using methanol (174A-220390) or acetic acid (174A-220391) as cosolvent.

FIG. 11 depicts cumulative release of OCT from microspheres with 15 wt. % target OCT loading prepared of 10LP2L20-GLL40 (3/97) via O/W membrane emulsification e using polymer concentrations of 10 wt. % (174A-220471) and 15 wt. % (174A-220495).

FIG. 12 depicts cumulative release of OCT from microspheres with 15 wt. % target OCT loading prepared of 10LP2L20-GLL40 (3/97) via O/W membrane emulsification using different batch size and extraction conditions.

FIG. 13 depicts cumulative release of OCT from microspheres with a target OCT loading of 10 wt. % (174A-230185) and 15 wt. % (174A-230186) prepared of 10LP2L20-GLL40 (3/97) at 5 g scale via the O/W membrane emulsification procedure.

FIG. 14 depicts cumulative release of OCT from microspheres with a target OCT loading of 10 wt. % (174A-2301 19) prepared of 10LP2L20-GLL40 (3/97) via membrane emulsification at 15 g scale.

### DETAILED DESCRIPTION

The present disclosure is based, in part, on the discovery of extended release octreotide dosage forms, kits of formulation, and methods that increase lifespan, promote longevity, and/or prevent, reduce the severity of, or delay the onset of various aging-associated conditions. The extended release octreotide dosage forms, kits of formulation, and methods further improve the lifespan of the mammal, which may include treating a cancer and/or preventing, reducing the severity of, or delaying the onset of the cancer in the mammal.

Reduced somatotropic [growth hormone (GH) and insulin-like growth factor-1 (IGF-1)] action has been shown to be associated with delayed aging, reduced age-related disease and functional decline, and with extended longevity. See, e.g., Berryman et al., “Role of the GH/IGF-1 axis in lifespan: lessons from animal models.” Growth Hormone & IGF Research 18.6 (2008): 455-471 and Bartke et al., “The somatotropic axis and aging: benefits of endocrine defects.” Growth Hormone & IGF Research 27 (2016): 41-45.

The literature describes that GH influences insulin/IGF-1 signaling (IIS) as a key stimulator of IGF-1 expression and is an important modulator of the secretion and actions of insulin. It has been reported that differences in lifespan among various mammalian species may also be related to somatotropic action (see, e.g., Fushan et al., “Gene expression defines natural changes in mammalian lifespan.” Aging Cell. 2015; 14:352-365).

Moreover, extended longevity in mice with hereditary defects in somatotropic (GH/IGF-1) action have been described (see, e.g., Brown-Borg et al., “Dwarf mice and the ageing process. Nature. 1996; 384:33; Flurkey K et al., “Lifespan extension and delayed immune and collagen aging in mutant mice with defects in growth hormone production.” Proc Natl Acad Sci USA. 2001; 98:6736-6741; and Coschigano et al., “Assessment of growth parameters and lifespan of GHR/BP gene-disrupted mice. Endocrinology. 2000; 141:2608-2613.).

Studies of the relationships between longevity and polymorphism of genes involved in GH and IGF-1 induced signaling have provided evidence that the somatotropic axis is involved in the control of aging in other mammalian species, including humans (see, e.g., Bartke et al., “Somatotropic signaling: trade-offs between growth, reproductive development, and longevity.” Physiol Rev. 2013; 93:571-598; Greer et al., “Connecting serum IGF-1, body size, and age in the domestic dog.” Age (Dordr) 2011; 33:475-483; van Heemst et al., “Reduced insulin/IGF-1 signalling and human longevity.” Aging Cell. 2005; 4:79-85; and Milman et al., “Low insulin-like growth factor-1 level predicts survival in humans with exceptional longevity.” Aging Cell. 2014; 13:769-771).

However, the relationship between somatotropic signaling and human aging is less striking, complex and controversial. Indeed, the interrelationships of GH actions and aging are complex and incompletely understood. (see Bartke and Darcy “GH and ageing: Pitfalls and new insights.” Best Practice & Research Clinical Endocrinology & Metabolism 31.1 (2017): 113-125.) Unfortunately, prior results focusing on GH as an anti-aging agent have been disappointing and with few documented benefits and many troublesome side effects (see, Bartke “Growth hormone and aging: updated review.” The world journal of men's health 37.1 (2019): 19-30.).

Accordingly, there is an unmet need for extended release octreotide dosage forms, kits of formulation, and methods that inhibit release of GH and increase lifespan, promote longevity, and/or prevent, reduce the severity of, or delay the onset of various aging-associated conditions. The extended release octreotide dosage forms and kits of formulation described herein of the present disclosure comprise a compound, octreotide, that inhibits release of GH.

### Compound

Octreotide (for example, brand name Sandostatin® (Novartis, NJ)) is an octapeptide that mimics natural somatostatin pharmacologically. Somatostatin, also known as growth hormone-inhibiting hormone (GHIH), is a peptide hormone that regulates the endocrine system via interaction with somatostatin receptors to inhibit release of numerous secondary hormones, e.g., GH, glucagon, and insulin. Notably, octreotide is a more potent inhibitor of GH, glucagon, and insulin than the natural hormone somatostatin.

Octreotide, as a somatostatin analog that inhibits the release of GH, is used for the treatment of GH-producing tumors (which cause acromegaly and gigantism), pituitary tumors that secrete thyroid-stimulating hormone (thyrotropinoma), and symptoms associated with carcinoid syndrome or in patients with vasoactive intestinal peptide-secreting tumors (VIPomas).

Octreotide is (4R,7S,10S,13R,16S,19R)-10-(4-aminobutyl)-19-[[(2R)-2-amino-3-phenyl-propanoyl]amino]-16-benzyl-N-[(2R,3R)-1,3-dihydroxybutan-2-yl]-7-(1-hydroxyethyl)-13-(1H-indol-3-ylmethyl)-6,9,12,15,18-pentaoxo-1,2-dithia-5,8,11,14,17-pentazacycloicosane-4-carboxamide. The molecular weight of octreotide free base is 1019.25. The structure of octreotide is:

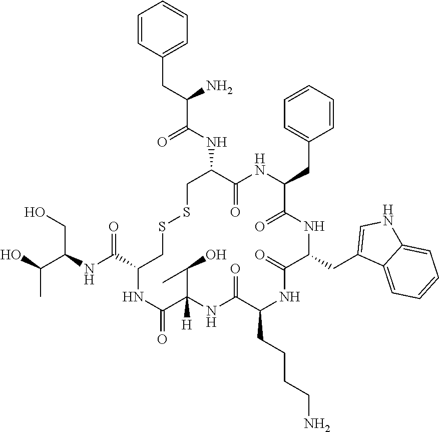 [SMILES: C[C@@H](O)[C@@H]1NC(=O)[C@H](CCCCN)NC(=O)[C@@H](Cc2c[nH]c3ccccc23)NC(=O)[C@H](Cc2ccccc2)NC(=O)[C@@H](NC(=O)[C@H](N)Cc2ccccc2)CSSC[C@@H](C(=O)N[C@H](CO)[C@@H](C)O)NC1=O]

In some embodiments disclosed herein, octreotide is in a salt form. In some embodiments disclosed herein, octreotide is an acetate salt. Octreotide acetate described herein comprises octreotide and two equivalents of acetic acid. The molecular weight of octreotide acetate is 1139.35.

### Dosage Forms

In one aspect, disclosed herein is an extended release octreotide dosage form comprising: (a) octreotide, or a pharmaceutically acceptable salt thereof; and (b) a biodegradable polymer microsphere. In some embodiments, the octreotide, or the pharmaceutically acceptable salt thereof, is present in the microsphere. In some embodiments, the microsphere provides for extended release of the octreotide, or the pharmaceutically acceptable salt thereof.

In one aspect, disclosed herein is an extended release octreotide dosage form comprising: (a) octreotide, or a pharmaceutically acceptable salt thereof; and (b) a biodegradable polymer microsphere, wherein the biodegradable polymer microsphere comprises about 10 wt % to 99 wt % of a biodegradable polymer, wherein the biodegradable polymer is a multiblock copolymer comprising at least one hydrolysable pre-polymer (A) segment and at least one hydrolysable pre-polymer (B) segment; wherein the segments are linked by a multifunctional chain extender; and wherein the octreotide, or the pharmaceutically acceptable salt thereof, is present in the microsphere.

In one aspect, disclosed herein is an extended release octreotide dosage form comprising: (a) octreotide, or a pharmaceutically acceptable salt thereof; (b) a biodegradable polymer microsphere; and (c) a diluent or injection vehicle; wherein the octreotide or the pharmaceutically acceptable salt thereof, is present in the biodegradable polymer microsphere, wherein the biodegradable polymer microsphere comprises about 10 wt % to 99 wt % of a biodegradable polymer, wherein the biodegradable polymer is a multiblock copolymer, wherein the biodegradable multiblock copolymer comprises a [poly(D,L-Lactide)-co-poly(ethylene glycol)-co-poly(D,L-Lactide)]-b-[poly(L-lactide-co-glycolide)] multiblock copolymer; and wherein the biodegradable multiblock copolymer is represented by [(R¹ₘ R²ₙ R³ₘ)ₚ]ᵣ[(R⁴)_{q})]ₛ, wherein: R¹ and R³ are each a poly(D,L-Lactide) segment; R² is a poly(ethylene glycol) segment; R⁴ is a poly(L-lactide-co-glycolide) segment, wherein the L-lactide:glycolide molar ratio is 99:1 to 80:20 mole/mole; m is a molecular weight of R¹ and R³, and m is 400 to 1000 g/mole; n is a molecular weight of R², and n is 100 to 1200 g/mole; p is a molecular weight of the (R¹ₘ R²ₙ R³ₘ) segment, and p is 1800 to 2200 g/mole; q is a molecular weight of the (R⁴) segment, and q is 3800 to 4200 g/mole; and r/s is a ratio of the (R¹ₘ R²ₙ R³ₘ) segment over the (R⁴), and r/s is 5/95 to 15/85 molar ratio. The molecular weight m of R¹ and R³ can be the same or different. In some embodiments, the biodegradable polymer comprises one [poly(D,L-Lactide)-co-poly(ethylene glycol)-co-poly(D,L-Lactide)] segment. In some embodiments, each of the [poly(D,L-Lactide)-co-poly(ethylene glycol)-co-poly(D,L-Lactide)] segment(s) in the biodegradable multiblock copolymer independently has a molecular weight p. In some embodiments, each of the (R¹ₘ R²ₙ R³ₘ) segment(s) in the biodegradable multiblock copolymer independently has a molecular weight p. In some embodiments, each of the [poly(L-lactide-co-glycolide)] segment(s) in the biodegradable multiblock copolymer independently has a molecular weight q. In some embodiments, each of the (R⁴) segment(s) in the biodegradable multiblock copolymer independently has a molecular weight q. In some embodiments, r/s is 5/95 to 10/90 molar ratio. In some embodiments, r/s is 10/90 to 15/85 molar ratio.

In some embodiments, the dosage form comprises about 5 wt % to about 50 wt % of the biodegradable polymer sphere. In some embodiments, the dosage form comprises about 5 wt % to about 40 wt % of the biodegradable polymer sphere. In some embodiments, the dosage form comprises about 5 wt % to about 30 wt % of the biodegradable polymer sphere. In some embodiments, the dosage form comprises about 5 wt % to about 20 wt % of the biodegradable polymer sphere. In some embodiments, the dosage form comprises about 10 wt % to about 20 wt % of the biodegradable polymer sphere.

In some embodiments, the dosage form comprises about 5 wt % to about 60 wt % of the biodegradable polymer microsphere. In some embodiments, the dosage form comprises about 5 wt % to about 60 wt % of the biodegradable polymer microsphere. In some embodiments, the dosage form comprises about 5 wt % to about 50 wt % of the biodegradable polymer microsphere. In some embodiments, the dosage form comprises about 5 wt % to about 40 wt % of the biodegradable polymer microsphere. In some embodiments, the dosage form comprises about 5 wt % to about 30 wt % of the biodegradable polymer microsphere. In some embodiments, the dosage form comprises about 5 wt % to about 20 wt % of the biodegradable polymer microsphere. In some embodiments, the dosage form comprises about 5 wt % to about 10 wt % of the biodegradable polymer microsphere. In some embodiments, the dosage form comprises about 10 wt % to about 20 wt % of the biodegradable polymer microsphere. In some embodiments, the dosage form comprises about 20 wt % to about 40 wt % of the biodegradable polymer microsphere. In some embodiments, the dosage form comprises about 15 wt % to about 50 wt % of the biodegradable polymer microsphere. In some embodiments, the dosage form comprises about 40 wt % to about 80 wt % of the biodegradable polymer microsphere.

In some embodiments, the dosage form comprises about 50 wt %, about 45 wt %, about 40 wt %, about 35 wt %, about 30 wt %, about 25 wt %, about 20 wt %, about 15 wt %, about 10 wt %, about 5 wt %, about 2 wt. %, or about 1 wt % of the octreotide, or a pharmaceutically acceptable salt thereof. In some embodiments, the dosage form comprises about 50 wt % of the octreotide, or a pharmaceutically acceptable salt thereof. In some embodiments, the dosage form comprises about 45 wt % of the octreotide, or a pharmaceutically acceptable salt thereof. In some embodiments, the dosage form comprises about 40 wt % of the octreotide, or a pharmaceutically acceptable salt thereof. In some embodiments, the dosage form comprises about 35 wt % of the octreotide, or a pharmaceutically acceptable salt thereof. In some embodiments, the dosage form comprises about 30 wt % of the octreotide, or a pharmaceutically acceptable salt thereof. In some embodiments, the dosage form comprises about 25 wt % of the octreotide, or a pharmaceutically acceptable salt thereof. In some embodiments, the dosage form comprises about 20 wt % of the octreotide, or a pharmaceutically acceptable salt thereof. In some embodiments, the dosage form comprises about 15 wt % of the octreotide, or a pharmaceutically acceptable salt thereof. In some embodiments, the dosage form comprises about 10 wt % of the octreotide, or a pharmaceutically acceptable salt thereof. In some embodiments, the dosage form comprises or about 5 wt % of the octreotide, or a pharmaceutically acceptable salt thereof.

In some embodiments, the microsphere comprises about 1 wt % to about 50 wt % of the octreotide, or the pharmaceutically acceptable salt thereof. In some embodiments, the microsphere comprises about 10 wt % to about 40 wt % of the octreotide, or the pharmaceutically acceptable salt thereof. In some embodiments, the microsphere comprises about 25 wt % to about 45 wt % of the octreotide, or the pharmaceutically acceptable salt thereof. In some embodiments, the microsphere comprises about 50 wt % to about 80 wt % of the octreotide, or the pharmaceutically acceptable salt thereof. In some embodiments, the microsphere comprises about 5 wt % to about 30 wt % of the octreotide, or the pharmaceutically acceptable salt thereof. In some embodiments, the neurosphere comprises about 2 wt % to about 10 wt % of the octreotide, or the pharmaceutically acceptable salt thereof. In some embodiments, the microsphere comprises about 1 wt % to about 5 wt % of the octreotide, or the pharmaceutically acceptable salt thereof. In some embodiments, the microsphere comprises about 10 wt % to about 20 wt % of the octreotide, or the pharmaceutically acceptable salt thereof. In some embodiments, the microsphere comprises about 10 wt % to about 15 wt % of the octreotide, or the pharmaceutically acceptable salt thereof. In some embodiments, the microsphere comprises about 10 wt % of the octreotide, or the pharmaceutically acceptable salt thereof. In some embodiments, the microsphere comprises about 15 wt % of the octreotide, or the pharmaceutically acceptable salt thereof.

In some embodiments, the microsphere comprises about 5 wt %, about 10 wt %, about 15 wt %, about 20 wt %, about 25 wt %, about 30 wt %, about 35 wt %, about 40 wt %, about 45 wt %, or about 50 wt % of the octreotide, or the pharmaceutically acceptable salt thereof. In some embodiments, the microsphere comprises about 10 wt % or about 15 wt % of the octreotide, or the pharmaceutically acceptable salt thereof. In some embodiments, the microsphere comprises about 5 wt % of the octreotide, or the pharmaceutically acceptable salt thereof. In some embodiments, the microsphere comprises about 10 wt % of the octreotide, or the pharmaceutically acceptable salt thereof. In some embodiments, the microsphere comprises about 15 wt % of the octreotide, or the pharmaceutically acceptable salt thereof. In some embodiments, the microsphere comprises about 20 wt % of the octreotide, or the pharmaceutically acceptable salt thereof. In some embodiments, the microsphere comprises about 25 wt % of the octreotide, or the pharmaceutically acceptable salt thereof. In some embodiments, the microsphere comprises about 30 wt % of the octreotide, or the pharmaceutically acceptable salt thereof. In some embodiments, the microsphere comprises about 35 wt % of the octreotide, or the pharmaceutically acceptable salt thereof. In some embodiments, the microsphere comprises about 40 wt % of the octreotide, or the pharmaceutically acceptable salt thereof. In some embodiments, the microsphere comprises about 45 wt % of the octreotide, or the pharmaceutically acceptable salt thereof. In some embodiments, the microsphere comprises about 50 wt % of the octreotide, or the pharmaceutically acceptable salt thereof.

In some embodiments, the microsphere is in a powder form. In some embodiments, the microsphere is in a lyophilized powder form. In some embodiments, the microsphere is in a powder form which is dried by lyophilization. In some embodiments, the microsphere is in a vacuum freeze-dried powder form. In some embodiments, the microsphere is in a powder form which is dried by vacuum freeze drying. In some embodiments, the microsphere is in a vacuum-dried powder form. In some embodiments, the microsphere is in a powder form which is dried in vacuo.

In some embodiments, the dosage form comprises about 1 mg to about 1000 mg of the octreotide, or a pharmaceutically acceptable salt thereof. In some embodiments, the dosage form comprises about 25 mg to about 500 mg of the octreotide, or a pharmaceutically acceptable salt thereof. In some embodiments, the dosage form comprises about 50 mg to about 250 mg of the octreotide, or a pharmaceutically acceptable salt thereof. In some embodiments, the dosage form comprises about 75 mg to about 150 mg of the octreotide, or a pharmaceutically acceptable salt thereof. In some embodiments, the dosage form comprises about 100 mg to about 500 mg of the octreotide, or a pharmaceutically acceptable salt thereof. In some embodiments, the dosage form comprises about 100 mg to about 200 mg of the octreotide, or a pharmaceutically acceptable salt thereof. In some embodiments, the dosage form comprises about 50 mg to about 250 mg of the octreotide, or a pharmaceutically acceptable salt thereof.

In some embodiments, a microsphere (or biodegradable polymer microsphere) described herein comprises about 30 wt % to about 95 wt % of a biodegradable polymer. In some embodiments, the microsphere comprises about 40 wt % to about 95 wt % of a biodegradable polymer. In some embodiments, the microsphere comprises about 50 wt % to about 95 wt % of a biodegradable polymer. In some embodiments, the microsphere comprises about 60 wt % to about 95 wt % of a biodegradable polymer. In some embodiments, the microsphere comprises about 70 wt % to about 95 wt % of a biodegradable polymer. In some embodiments, the microsphere comprises about 80 wt % to about 95 wt % of a biodegradable polymer. In some embodiments, the microsphere comprises about 80 wt % to about 90 wt % of a biodegradable polymer.

In some embodiments, a biodegradable polymer microsphere described herein comprises about 5 wt % to 99 wt % of a biodegradable polymer. In some embodiments, the biodegradable polymer microsphere comprises about 10 wt % to 99 wt % of a biodegradable polymer. In some embodiments, the biodegradable polymer microsphere comprises about 20 wt % to 95 wt % of a biodegradable polymer. In some embodiments, the biodegradable polymer microsphere comprises about 30 wt % to 95 wt % of a biodegradable polymer. In some embodiments, the biodegradable polymer microsphere comprises about 40 wt % to 95 wt % of a biodegradable polymer. In some embodiments, the biodegradable polymer microsphere comprises about 50 wt % to 95 wt % of a biodegradable polymer. In some embodiments, the biodegradable polymer microsphere comprises about 60 wt % to 95 wt % of a biodegradable polymer. In some embodiments, the biodegradable polymer microsphere comprises about 65 wt % to 95 wt % of a biodegradable polymer. In some embodiments, the biodegradable polymer microsphere comprises about 70 wt % to 95 wt % of a biodegradable polymer. In some embodiments, the biodegradable polymer microsphere comprises about 75 wt % to 95 wt % of a biodegradable polymer. In some embodiments, the biodegradable polymer microsphere comprises about 80 wt % to 95 wt % of a biodegradable polymer. In some embodiments, the biodegradable polymer microsphere comprises about 80 wt % to 90 wt % of a biodegradable polymer. In some embodiments, the biodegradable polymer microsphere comprises about 60 wt % to 80 wt % of a biodegradable polymer.

In some embodiments, a biodegradable polymer microsphere described herein comprises about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, or about 99 wt % of a biodegradable polymer. In some embodiments, the microsphere comprises about 30 wt % of a biodegradable polymer. In some embodiments, the microsphere comprises about 35 wt % of a biodegradable polymer. In some embodiments, the microsphere comprises about 40 wt % of a biodegradable polymer. In some embodiments, the microsphere comprises about 45 wt % of a biodegradable polymer. In some embodiments, the microsphere comprises about 50 wt % of a biodegradable polymer. In some embodiments, the microsphere comprises about 55 wt % of a biodegradable polymer. In some embodiments, the microsphere comprises about 60 wt % of a biodegradable polymer. In some embodiments, the microsphere comprises about 65 wt % of a biodegradable polymer. In some embodiments, the microsphere comprises about 70 wt % of a biodegradable polymer. In some embodiments, the microsphere comprises about 75 wt % of a biodegradable polymer. In some embodiments, the microsphere comprises about 80 wt % of a biodegradable polymer. In some embodiments, the microsphere comprises about 85 wt % of a biodegradable polymer. In some embodiments, the microsphere comprises about 90 wt % of a biodegradable polymer. In some embodiments, the neurosphere comprises about 95 wt % of a biodegradable polymer.

In some embodiments, a biodegradable polymer described herein comprises a biodegradable multiblock copolymer. In some embodiments, the biodegradable multiblock copolymer comprises at least one hydrolysable pre-polymer (A) segment and at least one hydrolysable pre-polymer (B) segment. In some embodiments, the biodegradable multiblock copolymer comprises at least one hydrolysable pre-polymer (A) segment. In some embodiments, the biodegradable multiblock copolymer comprises at least one hydrolysable pre-polymer (B) segment.

In some embodiments, the biodegradable polymer described herein is a biodegradable multiblock copolymer comprising one hydrolysable pre-polymer (A) segment and one hydrolysable pre-polymer (B) segment. In some embodiments, the biodegradable polymer described herein is a biodegradable multiblock copolymer comprising one hydrolysable pre-polymer (A) segment. In some embodiments, the biodegradable polymer described herein is a biodegradable multiblock copolymer comprising one hydrolysable pre-polymer (B) segment.

In some embodiments, the prepolymer (A) segment and/or the pre-polymer (B) segment comprises one or more linkages selected from the group consisting of ester linkages, carbonate linkages, anhydride linkages, ether linkages, and combinations thereof. In some embodiments, the prepolymer (A) segment comprises one or more polyether groups. In some embodiments, the one or more polyether groups are selected from the group consisting of: polyethylene glycol, polyethylene glycol-polypropylene glycol, polytetramethylene ether glycol, and combinations thereof. In some embodiments, the polyether group is polyethylene glycol. In some embodiments, a polyether is present as an additional prepolymer in the multiblock copolymer.

In some embodiments, the prepolymer (A) segment comprises products of a reaction of at least one cyclic monomer with at least one noncyclic initiator selected from the group consisting of diols, dicarboxylic acids, and hydroxycarboxylic acids. In some embodiments, the at least one cyclic monomer is selected from the group consisting of glycolide, lactide (D and/or L), e-caprolactone, d-valerolactone, trimethylene carbonate, 1,4-dioxan-2-one (para-dioxanone), 1,5-dioxan-2-one, and a cyclic anhydride In some embodiments, the at least one noncyclic initiator is selected from the group consisting of succinic acid, glutaric acid, adipic acid, sebacic acid, lactic acid, glycolic acid, ethylene glycol, diethylene glycol, 1,4-butanediol, and 1,6-hexanediol.

In some embodiments, the prepolymer (A) segment comprises a reaction product of ester forming monomers selected from diols, dicarboxylic acids, and hydroxycarboxylic acids. In some embodiments, the prepolymer (A) segment comprises reaction products of glycolide, lactide (D and/or L), e-caprolactone, and/or d-valerolactone.

In some embodiments, the prepolymer (A) segment comprises a reaction product of initiators selected from diols, dicarboxylic acids, or hydroxycarboxylic acids. In some embodiments, the prepolymer (A) segment comprises a reaction product of ester forming monomers selected from glycolide, lactide (D and/or L), e-caprolactone, and d-valerolactone.

In some embodiments, the amount of prepolymer (A) in the multiblock copolymer is from about 1% to about 90% based on total weight of the multiblock copolymer. In some embodiments, the prepolymer (A) segment has a Mn of about 500 g/mol or more.

In some embodiments, the prepolymer (B) segment comprises a polymer derived from hydroxyalkanoate, glycolide, lactide (D and/or L), ε-caprolactone, δ-valerolactone, trimethylene carbonate, 1,4-dioxan-2-one or combinations thereof.

In some embodiments, the prepolymer (B) segment comprises poly(L-lactide-co-glycolide). In some embodiments, the prepolymer (B) segment comprises poly(L-lactide-co-glycolide) with a Mn of about 1000 g/mol or more. In some embodiments, the prepolymer (B) segment comprises poly(L-lactide-co-glycolide) with a Mn of about 2000 g/mol or more. In some embodiments, the prepolymer (B) segment comprises poly(L-lactide-co-glycolide) with a Mn of about 3000 g/mol or more. In some embodiments, the prepolymer (B) segment comprises poly(L-lactide-co-glycolide) with a Mn of about 4000 g/mol.

In some embodiments, the at least one hydrolysable pre-polymer (B) segment comprises poly(L-lactide-co-glycolide) with a glycolide/L-lactide monomer molar ratio of about 0.01 or more, 0.02 or more, about 0.03 or more, 0.04 or more, about 0.05 or more, 0.06 or more, 0.07 or more, 0.08 or more, 0.09 or more, about 0.1 or more, about 0.2 or more, about 0.3 or more, about 0.4 or more, about 0.5 or more, about 0.6 or more, about 0.7 or more, about 0.8 or more, about 0.9 or more, or about 1.0 or more. In some embodiments, the at least one hydrolysable pre-polymer (B) segment comprises poly(L-lactide-co-glycolide) with a glycolide/L-lactide monomer molar ratio of about 0.01 or more, 0.02 or more, about 0.03 or more, 0.04 or more, about 0.05 or more, 0.06 or more, 0.07 or more, 0.08 or more, 0.09 or more, about 0.1 or more, about 0.2 or more, about 0.3 or more, about 0.4 or more, about 0.5 or more, or about 1.0 or more. In some embodiments, the at least one hydrolysable pre-polymer (B) segment comprises poly(lactide-co-glycolide) with a glycolide/lactide monomer molar ratio of about 1:100 to 100:1, 1:100 to 1:10, 1:100 to 1:1, 1:20 to 1:1, 1:20 to 20:1, 1:1 to 100:1, 1:5 to 5:1, or 100:1 to 10:1. In some embodiments, the at least one hydrolysable pre-polymer (B) segment comprises poly(lactide-co-glycolide) with a glycolide/lactide monomer molar ratio of about 1:30 to 1:3. In some embodiments, the at least one hydrolysable pre-polymer (B) segment comprises poly(lactide-co-glycolide) with a glycolide/lactide monomer molar ratio of about 1:20 to 1:4.

In some embodiments, the at least one hydrolysable pre-polymer (B) segment comprises poly(L-lactide-co-glycolide) with a glycolide/L-lactide monomer molar ratio of about 0.01 or more, about 0.03 or more, about 0.05 or more, about 0.1 or more, about 0.2 or more, about 0.3 or more, about 0.4 or more, about 0.5 or more, or about 1.0 or more.

In some embodiments, the at least one hydrolysable pre-polymer (B) segment comprises poly(L-lactide-co-glycolide) with a glycolide/L-lactide monomer molar ratio of about 0.01 or more. In some embodiments, the at least one hydrolysable pre-polymer (B) segment comprises poly(L-lactide-co-glycolide) with a glycolide/L-lactide monomer molar ratio of about 0.02 or more. In some embodiments, the at least one hydrolysable pre-polymer (B) segment comprises poly(L-lactide-co-glycolide) with a glycolide/L-lactide monomer molar ratio of about 0.03 or more. In some embodiments, the at least one hydrolysable pre-polymer (B) segment comprises poly(L-lactide-co-glycolide) with a glycolide/L-lactide monomer molar ratio of about 0.04 or more. In some embodiments, the at least one hydrolysable pre-polymer (B) segment comprises poly(L-lactide-co-glycolide) with a glycolide/L-lactide monomer molar ratio of about 0.05 or more. N some embodiments, the at least one hydrolysable pre-polymer (B) segment comprises poly(L-lactide-co-glycolide) with a glycolide/L-lactide monomer molar ratio of about 0.06 or more. N some embodiments, the at least one hydrolysable pre-polymer (B) segment comprises poly(L-lactide-co-glycolide) with a glycolide/L-lactide monomer molar ratio of about 0.07 or more. N some embodiments, the at least one hydrolysable pre-polymer (B) segment comprises poly(L-lactide-co-glycolide) with a glycolide/L-lactide monomer molar ratio of about 0.08 or more. N some embodiments, the at least one hydrolysable pre-polymer (B) segment comprises poly(L-lactide-co-glycolide) with a glycolide/L-lactide monomer molar ratio of about 0.09 or more. In some embodiments, the at least one hydrolysable pre-polymer (B) segment comprises poly(L-lactide-co-glycolide) with a glycolide/L-lactide monomer molar ratio of about 0.1 or more. In some embodiments, the at least one hydrolysable pre-polymer (B) segment comprises poly(L-lactide-co-glycolide) with a glycolide/L-lactide monomer molar ratio of about 0.2 or more. In some embodiments, the at least one hydrolysable pre-polymer (B) segment comprises poly(L-lactide-co-glycolide) with a glycolide/L-lactide monomer molar ratio of about 0.3 or more. In sone embodiments, the at least one hydrolysable pre-polymer (B) segment comprises poly(L-lactide-co-glycolide) with a glycolide/L-lactide monomer molar ratio of about 0.4 or more. In some embodiments, the at least one hydrolysable pre-polymer (B) segment comprises poly(L-lactide-co-glycolide) with a glycolide/L-lactide monomer molar ratio of about 0.5 or more. In some embodiments, the at least one hydrolysable pre-polymer (B) segment comprises poly(L-lactide-co-glycolide) with a glycolide/L-lactide monomer molar ratio of about 0.6 or more. In some embodiments, the at least one hydrolysable pre-polymer (B) segment comprises poly(L-lactide-co-glycolide) with a glycolide/L-lactide monomer molar ratio of about 0.7 or more. In some embodiments, the at least one hydrolysable pre-polymer (B) segment comprises poly(L-lactide-co-glycolide) with a glycolide/L-lactide monomer molar ratio of about 0.8 or more. In some embodiments, the at least one hydrolysable pre-polymer (B) segment comprises poly(L-lactide-co-glycolide) with a glycolide/L-lactide monomer molar ratio of about 0.9 or more. In some embodiments, the at least one hydrolysable pre-polymer (B) segment comprises poly(L-lactide-co-glycolide) with a glycolide/L-lactide monomer molar ratio of about 1.0 or more.

In some embodiments, the at least one hydrolysable pre-polymer (B) segment is poly(L-lactide-co-glycolide) with a with a glycolide/L-lactide monomer molar ratio of 3/97.

In some embodiments, the at least one hydrolysable pre-polymer (B) segment is poly(L-lactide-co-glycolide) with a with a glycolide/L-lactide monomer molar ratio of 15/85.

In some embodiments, the prepolymer (B) segment comprises a molar amount of about 2 wt % to about 25 wt % of glycolide relative to the combined molar amount of glycolide and L-lactide.

In some embodiments, the prepolymer (B) segment comprises poly(p-dioxanone). In some embodiments, the prepolymer (B) segment comprises poly(p-dioxanone) with a Mn of about 1000 g/mol or more. In some embodiments, the prepolymer (B) segment comprises poly(/i-dioxanone) with a Mn of about 1500 g/mol or more. In some embodiments, the prepolymer (B) segment comprises poly(p-dioxanone) with a Mn of about 2000 g/mol or more. In some embodiments, the prepolymer (13) segment comprises poly(p-dioxanone) with a Mn of about 2500 g/mol or more. In some embodiments, the prepolymer (B) segment comprises poly(/i-dioxanone) with a Min of about 3000 g/mol or more. In some embodiments, the prepolymer (B) segment comprises poly(p-dioxanone) with a Mn of about 3500 g/mol or more. In some embodiments, the prepolymer (B) segment comprises poly(p-dioxanone) with a Mn of about 4000 g/mol or more,

In some embodiments, the multiblock copolymer comprises from about 10 wt % to about 99 wt % of the prepolymer (B) segment relative to the total weight of the multiblock copolymer.

In some embodiments, the segments are linked by a multifunctional chain extender. In some embodiments, the multifunctional chain extender is a difunctional aliphatic chain extender. In some embodiments, the multifunctional chain extender is a diisocyanate. In some embodiments, the diisocyanate is 1,4-butanediisocyanate.

In some embodiments, the biodegradable multiblock copolymer has a T_{g} of 37° C. or less and a Tₘ of 110-250° C. In some embodiments, the biodegradable multiblock copolymer has a T_{g} of 37° C. or less. In some embodiments, the biodegradable multiblock copolymer has a Tₘ of 110-250° C.

In some embodiments, the segments are randomly distributed over the chain of the biodegradable multiblock copolymer.

In some embodiments, the amount of at least one hydrolysable pre-polymer (A) segment in the copolymer is about 10% to about 90% based on total weight of the biodegradable multiblock copolymer.

In some embodiments, the amount of the hydrolysable pre-polymer (A) segment in the biodegradable multiblock copolymer is about 5% to about 90% based on total weight of the biodegradable multiblock copolymer. In some embodiments, the amount of the hydrolysable pre-polymer (A) segment in the biodegradable multiblock copolymer is about 5% to about 80% based on total weight of the biodegradable multiblock copolymer. In some embodiments, the amount of the hydrolysable pre-polymer (A) segment in the biodegradable multiblock copolymer is about 5% to about 70% based on total weight of the biodegradable multiblock copolymer. In some embodiments, the amount of the hydrolysable pre-polymer (A) segment in the biodegradable multiblock copolymer is about 5% to about 60% based on total weight of the biodegradable multiblock copolymer. In some embodiments, the amount of the hydrolysable pre-polymer (A) segment in the biodegradable multiblock copolymer is about 5% to about 50% based on total weight of the biodegradable multiblock copolymer. In some embodiments, the amount of the hydrolysable pre-polymer (A) segment in the biodegradable multiblock copolymer is about 5% to about 40% based on total weight of the biodegradable multiblock copolymer. In some embodiments, the amount of the hydrolysable pre-polymer (A) segment in the biodegradable multiblock copolymer is about 5% to about 30% based on total weight of the biodegradable multiblock copolymer. In some embodiments, the amount of the hydrolysable pre-polymer (A) segment in the biodegradable multiblock copolymer is about 5% to about 20% based on total weight of the biodegradable multiblock copolymer. In some embodiments, the amount of the hydrolysable pre-polymer (A) segment in the biodegradable multiblock copolymer is about 5% to about 10% based on total weight of the biodegradable multiblock copolymer.

In some embodiments, the amount of the hydrolysable pre-polymer (A) segment in the biodegradable multiblock copolymer is about 90% based on total weight of the biodegradable multiblock copolymer. In some embodiments, the content of at least one hydrolysable pre-polymer (A) segment in the biodegradable multiblock copolymer is about 80% based on total weight of the biodegradable multiblock copolymer. In some embodiments, the amount of the hydrolysable pre-polymer (A) segment in the biodegradable multiblock copolymer is about 70% based on total weight of the biodegradable multiblock copolymer. In some embodiments, the amount of the hydrolysable pre-polymer (A) segment in the biodegradable multiblock copolymer is about 60% based on total weight of the biodegradable multiblock copolymer. In some embodiments, the amount of the hydrolysable pre-polymer (A) segment in the biodegradable multiblock copolymer is about 50% based on total weight of the biodegradable multiblock copolymer. In some embodiments, the amount of the hydrolysable pre-polymer (A) segment in the biodegradable multiblock copolymer is about 40% based on total weight of the biodegradable multiblock copolymer. In some embodiments, the amount of the hydrolysable pre-polymer (A) segment in the biodegradable multiblock copolymer is about 30% based on total weight of the biodegradable multiblock copolymer. In some embodiments, the content of at least one hydrolysable pre-polymer (A) segment in the biodegradable multiblock copolymer is about 20% based on total weight of the biodegradable multiblock copolymer. In some embodiments, the amount of the hydrolysable pre-polymer (A) segment in the biodegradable multiblock copolymer is about 10% based on total weight of the biodegradable multiblock copolymer. In some embodiments, the amount of the hydrolysable pre-polymer (A) segment in the biodegradable multiblock copolymer is about 5% based on total weight of the biodegradable multiblock copolymer.

In some embodiments, the at least one hydrolysable pre-polymer (A) segment comprises poly(ethylene glycol) (PEG).

In some embodiments, the at least one hydrolysable pre-polymer (A) segment comprises reaction products of ester forming monomers selected from diols, dicarboxylic acids, and hydroxycarboxylic acids.

In some embodiments, the at least one hydrolysable pre-polymer (A) segment comprises reaction products of initiators selected from diols, dicarboxylic acids, or hydroxycarboxylic acids.

In some embodiments, the at least one hydrolysable pre-polymer (A) segment comprises reaction products of ester forming monomers selected from ethylene glycol, poly(ethylene glycol) (PEG), glycolide, lactide (D and/or L), ε-caprolactone, or δ-valerolactone. In some embodiments, the at least one hydrolysable pre-polymer (A) segment comprises a reaction product of poly(ethylene glycol) (PEG) and ε-caprolactone.

In some embodiments, the at least one hydrolysable pre-polymer (A) segment comprises a reaction product of ester forming monomers selected from glycolide, lactide (D and/or L), ε-caprolactone, and δ-valerolactone.

In some embodiments, the at least one hydrolysable pre-polymer (A) segment comprises a reaction product of poly(ethylene glycol) (PEG) and D,L-lactide.

In some embodiments, the at least one hydrolysable pre-polymer (A) segment is poly(D,L-lactide)-co-poly(ethylene glycol)-co-poly(D,L-lactide).

In some embodiments, the amount of the hydrolysable pre-polymer (A) in the biodegradable multiblock copolymer is from about 10% to about 90% based on total weight of the biodegradable multiblock copolymer. In some embodiments, the amount of the at least one hydrolysable pre-polymer (A) in the biodegradable multiblock copolymer is about 5 wt % to about 20 wt %, about 5 wt % to about 30 wt %, about 5 wt % to about 40 wt %, about 15 wt % to about 50 wt %, about 5 wt % to about 60 wt %, about 5 wt % to about 70 wt %, about 5 wt % to about 80 wt %, about 5 wt % to about 90 wt %, about 10 wt % to about 20 wt %, about 10 wt % to about 30 wt %, about 10 wt % to about 40 wt %, about 10 wt % to about 50 wt %, about 10 wt % to about 60 wt %, about 10 wt % to about 70 wt %, about 10 wt % to about 80 wt %, about 10 wt % to about 90 wt %, about 20 wt % to about 30 wt %, about 20 wt % to about 40 wt %, about 20 wt % to about 50 wt %, about 20 wt % to about 60 wt %, about 20 wt % to about 70 wt %, about 20 wt % to about 80 wt %, about 20 wt % to about 90 wt %, about 30 wt % to about 40 wt %, about 30 wt % to about 50 wt %, about 30 wt % to about 60 wt %, about 30 wt % to about 70 wt %, about 30 wt % to about 80 wt %, about 30 wt % to about 90 wt %, about 40 wt % to about 50 wt %, about 40 wt % to about 60 wt %, about 40 wt % to about 70 wt %, about 40 wt % to about 80 wt %, about 40 wt % to about 90 wt %, about 50 wt % to about 60 wt %, about 50 wt % to about 70 wt %, about 50 wt % to about 80 wt %, about 50 wt % to about 90 wt %, about 60 wt % to about 70 wt %, about 60 wt % to about 80 wt %, about 60 wt % to about 90 wt %, about 70 wt % to about 80 wt %, about 70 wt % to about 90 wt %, or about 80 wt % to about 90 wt % based on total weight of the biodegradable multiblock copolymer. In some embodiments, the amount of the at least one hydrolysable pre-polymer (A) in the biodegradable multiblock copolymer is about 5 wt %, about 10 wt %, about 20 wt %, about 30 wt %, about 40 wt %, about 50 wt %, about 60 wt %, about 70 wt %, about 80 wt %, or about 90 wt % based on total weight of the biodegradable multiblock copolymer. In some embodiments, the amount of the hydrolysable pre-polymer (A) in the biodegradable multiblock copolymer is at least about 5 wt %, about 10 wt %, about 20 wt %, about 30 wt %, about 40 wt %, about 50 wt %, about 60 wt %, about 70 wt %, or about 80 wt % based on total weight of the biodegradable multiblock copolymer. In some embodiments, the amount of the at least one hydrolysable pre-polymer (A) in the biodegradable multiblock copolymer is at most about 20 wt %, about 30 wt %, about 40 wt %, about 50 wt %, about 60 wt %, about 70 wt %, about 80 wt %, or about 90 wt % based on total weight of the biodegradable multiblock copolymer. In some embodiments, the at least one hydrolysable pre-polymer (A) segment has a Mₙ of about 500 g/mol or more, about 700 g/mol or more, about 1000 g/mol or more, 1200 g/mol or more, about 2000 g/mol or more, about 3000 g/mol or more, or about 4000 g/mol or more. In some embodiments, the at least one hydrolysable pre-polymer (A) segment has a Mₙ of about 500 g/mol or more, about 700 g/mol or more, about 1000 g/mol or more, about 2000 g/mol or more, about 3000 g/mol or more, or about 4000 g/mol or more. In some embodiments, the at least one hydrolysable pre-polymer (A) segment has a Mₙ of about 1200 g/mol or more or about 2000 g/mol or more. In some embodiments, the at least one hydrolysable pre-polymer (A) segment has a Mₙ of about 500 g/mol or more. In some embodiments, the at least one hydrolysable pre-polymer (A) segment has a Mₙ of about 700 g/mol or more. In some embodiments, the at least one hydrolysable pre-polymer (A) segment has a Mₙ of about 1000 g/mol or more. In some embodiments, the at least one hydrolysable pre-polymer (A) segment has a Mₙ of about 1200 g/mol or more. In some embodiments, the at least one hydrolysable pre-polymer (A) segment has a Mₙ of about 2000 g/mol or more. In some embodiments, the at least one hydrolysable pre-polymer (A) segment has a Mₙ of about 3000 g/mol or more. In some embodiments, the at least one hydrolysable pre-polymer (A) segment has a Mₙ of or about 4000 g/mol or more.

In some embodiments, the at least one hydrolysable pre-polymer (B) segment comprises reaction products of diols and lactide (D and/or L).

In some embodiments, the at least one hydrolysable pre-polymer (B) segment comprises reaction products of diols, L-lactide, and glycolide.

In some embodiments, the amount of the at least one hydrolysable pre-polymer (B) segment in the copolymer is about 10% to about 90% based on total weight of the biodegradable multiblock copolymer. In some embodiments, the amount of the hydrolysable pre-polymer (B) segment in the copolymer is about 10 wt % to about 20 wt %, about 10 wt % to about 30 wt %, about 10 wt % to about 40 wt %, about 10 wt % to about 50 wt %, about 10 wt % to about 60 wt %, about 10 wt % to about 70 wt %, about 10 wt % to about 80 wt %, about 10 wt % to about 90 wt %, about 20 wt % to about 30 wt %, about 20 wt % to about 40 wt %, about 20 wt % to about 50 wt %, about 20 wt % to about 60 wt %, about 20 wt % to about 70 wt %, about 20 wt % to about 80 wt %, about 20 wt % to about 90 wt %, about 30 wt % to about 40 wt %, about 30 wt % to about 50 wt %, about 30 wt % to about 60 wt %, about 30 wt % to about 70 wt %, about 30 wt % to about 80 wt %, about 30 wt % to about 90 wt %, about 40 wt % to about 50 wt %, about 40 wt % to about 60 wt %, about 40 wt % to about 70 wt %, about 40 wt % to about 80 wt %, about 40 wt % to about 90 wt %, about 50 wt % to about 60 wt %, about 50 wt % to about 70 wt %, about 50 wt % to about 80 wt %, about 50 wt % to about 90 wt %, about 60 wt % to about 70 wt %, about 60 wt % to about 80 wt %, about 60 wt % to about 90 wt %, about 70 wt % to about 80 wt %, about 70 wt % to about 90 wt %, or about 80 wt % to about 90 wt % based on total weight of the biodegradable multiblock copolymer. In some embodiments, the amount of the hydrolysable pre-polymer (B) segment in the copolymer is about 10 wt %, about 20 wt %, about 30 wt %, about 40 wt %, about 50 wt %, about 60 wt %, about 70 wt %, about 80 wt %, or about 90 wt % based on total weight of the biodegradable multiblock copolymer. In some embodiments, the amount of the hydrolysable pre-polymer (B) segment in the copolymer is at least about 10 wt %, about 20 wt %, about 30 wt %, about 40 wt %, about 50 wt %, about 60 wt %, about 70 wt %, or about 80 wt % based on total weight of the biodegradable multiblock copolymer. In some embodiments, the amount of the hydrolysable pre-polymer (B) segment in the copolymer is at most about 20 wt %, about 30 wt %, about 40 wt %, about 50 wt %, about 60 wt %, about 70 wt %, about 80 wt %, or about 90 wt % based on total weight of the biodegradable multiblock copolymer.

In some embodiments, the amount of the hydrolysable pre-polymer (B) segment in the biodegradable multiblock copolymer is about 40% to about 95% based on total weight of the biodegradable multiblock copolymer. In some embodiments, the amount of the hydrolysable pre-polymer (B) segment in the biodegradable multiblock copolymer is about 50% to about 95% based on total weight of the biodegradable multiblock copolymer. In some embodiments, the amount of the hydrolysable pre-polymer (B) segment in the biodegradable multiblock copolymer is about 60% to about 95% based on total weight of the biodegradable multiblock copolymer. In some embodiments, the amount of the hydrolysable pre-polymer (B) segment in the biodegradable multiblock copolymer is about 70% to about 95% based on total weight of the biodegradable multiblock copolymer. In some embodiments, the amount of the hydrolysable pre-polymer (B) segment in the biodegradable multiblock copolymer is about 80% to about 95% based on total weight of the biodegradable multiblock copolymer. In some embodiments, the amount of the hydrolysable pre-polymer (B) segment in the biodegradable multiblock copolymer is about 85% to about 95% based on total weight of the biodegradable multiblock copolymer.

In some embodiments, the amount of the hydrolysable pre-polymer (B) segment in the biodegradable multiblock copolymer is about 40% based on total weight of the biodegradable multiblock copolymer. In some embodiments, the amount of the hydrolysable pre-polymer (B) segment in the biodegradable multiblock copolymer is about 45% based on total weight of the biodegradable multiblock copolymer. In some embodiments, the amount of the hydrolysable pre-polymer (B) segment in the biodegradable multiblock copolymer is about 50% based on total weight of the biodegradable multiblock copolymer. In some embodiments, the amount of the hydrolysable pre-polymer (B) segment in the biodegradable multiblock copolymer is about 55% based on total weight of the biodegradable multiblock copolymer. In some embodiments, the amount of the hydrolysable pre-polymer (B) segment in the biodegradable multiblock copolymer is about 60% based on total weight of the biodegradable multiblock copolymer. In some embodiments, the amount of the hydrolysable pre-polymer (B) segment in the biodegradable multiblock copolymer is about 65% based on total weight of the biodegradable multiblock copolymer. In some embodiments, the amount of the hydrolysable pre-polymer (B) segment in the biodegradable multiblock copolymer is about 70% based on total weight of the biodegradable multiblock copolymer. In some embodiments, the amount of the hydrolysable pre-polymer (B) segment in the biodegradable multiblock copolymer is about 75% based on total weight of the biodegradable multiblock copolymer. In some embodiments, the amount of the hydrolysable pre-polymer (B) segment in the biodegradable multiblock copolymer is about 80% based on total weight of the biodegradable multiblock copolymer. In some embodiments, the amount of the hydrolysable pre-polymer (B) segment in the biodegradable multiblock copolymer is about 85% based on total weight of the biodegradable multiblock copolymer. In some embodiments, the amount of the hydrolysable pre-polymer (B) segment in the biodegradable multiblock copolymer is about 90% based on total weight of the biodegradable multiblock copolymer. In some embodiments, the amount of the hydrolysable pre-polymer (B) segment in the biodegradable multiblock copolymer is about 95% based on total weight of the biodegradable multiblock copolymer.

In some embodiments, the at least one hydrolysable pre-polymer (B) segment comprises poly(L-lactide).

In some embodiments, the at least one hydrolysable pre-polymer (B) segment comprises poly(L-lactide) with a Mₙ of about 1000 g/mol or more, about 2000 g/mol or more, about 3000 g/mol or more, or about 4000 g/mol or more.

In some embodiments, the at least one hydrolysable pre-polymer (B) segment comprises poly(L-lactide) with a Mₙ of about 1000 g/mol or more. In some embodiments, the at least one hydrolysable pre-polymer (B) segment comprises poly(L-lactide) with a Mₙ of about 2000 g/mol or more. In some embodiments, the at least one hydrolysable pre-polymer (B) segment comprises poly(L-lactide) with a Mₙ of about 3000 g/mol or more. In some embodiments, the at least one hydrolysable pre-polymer (B) segment comprises poly(L-lactide) with a Mₙ of or about 4000 g/mol or more.

In some embodiments, the at least one hydrolysable pre-polymer (B) segment comprises poly(L-lactide-co-glycolide).

In some embodiments, the at least one hydrolysable pre-polymer (B) segment comprises poly(L-lactide-co-glycolide) with a M₁ of about 500 g/mol or more, about 1000 g/mol or more, about 1200 g/mol or more, about 1500 g/mol or more, about 2000 g/mol or more, about 2500 g/mol or more, about 3000 g/mol or more, about 3500 g/mol or more, about 4000 g/mol or more, about 4500 g/mol or more, or about 5000 g/mol or more. In some embodiments, the at least one hydrolysable pre-polymer (B) segment comprises poly(L-lactide-co-glycolide) with a Mₙ of about 1000 g/mol or more, about 1200 g/mol or more, about 2000 g/mol or more, about 3000 g/mol or more, or about 4000 g/mol or more. In some embodiments, the at least one hydrolysable pre-polymer (B) segment comprises poly(L-lactide-co-glycolide) with a Mₙ of about 3000 g/mol or more or about 4000 g/mol or more.

In some embodiments, the at least one hydrolysable pre-polymer (B) segment comprises poly(L-lactide-co-glycolide) with a Mₙ of about 500 g/mol or more. In some embodiments, the at least one hydrolysable pre-polymer (B) segment comprises poly(L-lactide-co-glycolide) with a Mₙ of about 1000 g/mol or more. In some embodiments, the at least one hydrolysable pre-polymer (B) segment comprises poly(L-lactide-co-glycolide) with a Mₙ of about 1200 g/mol or more. In some embodiments, the at least one hydrolysable pre-polymer (B) segment comprises poly(L-lactide-co-glycolide) with a Mₙ of about 1500 g/mol or more. In some embodiments, the at least one hydrolysable pre-polymer (B) segment comprises poly(L-lactide-co-glycolide) with a Mₙ of about 2000 g/mol or more. In some embodiments, the at least one hydrolysable pre-polymer (B) segment comprises poly(L-lactide-co-glycolide) with a Mₙ of about 2500 g/mol or more. In some embodiments, the at least one hydrolysable pre-polymer (B) segment comprises poly(L-lactide-co-glycolide) with a Mₙ of about 3000 g/mol or more. In some embodiments, the at least one hydrolysable pre-polymer (B) segment comprises poly(L-lactide-co-glycolide) with a Mₙ of about 3500 g/mol or more. In some embodiments, the at least one hydrolysable pre-polymer (B) segment comprises poly(L-lactide-co-glycolide) with a Mₙ of or about 4000 g/mol or more.

In some embodiments, the poly(ethylene glycol) (PEG) has a Mₙ of about 150 to about 5000 g/mol, about 200 g/mol to about 1500 g/mol, about 600 to about 1000 g/mol, about 400 to about 3000 g/mol, about 600 to about 1500 g/mol, about 600 to about 5000 g/mol, or about 1000 to about 3000 g/mol. In some embodiments, the poly(ethylene glycol) (PEG) has a M₁ of about 150 to about 5000 g/mol. In some embodiments, the poly(ethylene glycol) (PEG) has a Mₙ of about 200 g/mol to about 1500 g/mol. In some embodiments, the poly(ethylene glycol) (PEG) has a Mₙ of about 600 to about 1000 g/mol. In some embodiments, the poly(ethylene glycol) (PEG) has a Mₙ of about 400 to about 3000 g/mol. In some embodiments, the poly(ethylene glycol) (PEG) has a M₁ of about 600 to about 1500 g/mol. In some embodiments, the poly(ethylene glycol) (PEG) has a Mₙ of about 600 to about 5000 g/mol. In some embodiments, the poly(ethylene glycol) (PEG) has a M₁ of or about 1000 to about 3000 g/mol.

In some embodiments, the biodegradable polymer microsphere has a swelling ratio under physiological conditions of about 1 to about 5, about 1 to about 4.5, about 1 to about 4, about 1 to about 3.5, about 1 to about 3, about 1 to about 2.5, about 1 to about 2, or about 1 to about 1.5. In some embodiments, the biodegradable polymer microsphere has a swelling ratio under physiological conditions of about 1 to about 4, about 1 to about 2, or about 1 to about 1.5. In some embodiments, the biodegradable polymer microsphere has a swelling ratio under physiological conditions of about 1 to about 5. In some embodiments, the biodegradable polymer microsphere has a swelling ratio under physiological conditions of about 1 to about 4.5. In some embodiments, the biodegradable polymer microsphere has a swelling ratio under physiological conditions of about 1 to about 4. In some embodiments, the biodegradable polymer microsphere has a swelling ratio under physiological conditions of about 1 to about 3.5. In some embodiments, the biodegradable polymer microsphere has a swelling ratio under physiological conditions of about 1 to about 3. In some embodiments, the biodegradable polymer microsphere has a swelling ratio under physiological conditions of about 1 to about 2.5. In some embodiments, the biodegradable polymer microsphere a swelling ratio under physiological conditions of about 1 to about 2. In some embodiments, the biodegradable polymer microsphere has a swelling ratio under physiological conditions of or about 1 to about 1.5.

In some embodiments, the biodegradable multiblock copolymer comprises a [poly(ε-caprolactone)-co-poly(ethylene glycol)-co-poly(ε-caprolactone)]-b-[poly(L-lactide)] multiblock copolymer.

In some embodiments, the biodegradable multiblock copolymer comprises a [poly(D,L-lactide)-co-poly(ethylene glycol)-co-poly(D,L-lactide)]-b-[poly(L-lactide-co-glycolide)] multiblock copolymer.

In some embodiments, the biodegradable multiblock copolymer as represented by [(R¹ₘ R²ₙ R³ₘ)ₚ]ᵣ[(R⁴)_{q})]ₛ is a [poly(D,L-Lactide)-co-poly(ethylene glycol)-co-poly(D,L-Lactide)]-b-[poly(L-lactide-co-glycolide)] multiblock copolymer wherein the L-lactide:glycolide molar ratio of R⁴ is 90:10 to 80:20 mole/mole, m is 400-600 g/mole, n is 800-1200 g/mole, p is 1800-2200 g/mole, q is 3800-4200 g/mole, and r/s is 5/95-15/85 molar ratio.

In some embodiments, the biodegradable multiblock copolymer as represented by [(R¹ₘ R²ₙ R³ₘ)ₚ]ᵣ[(R⁴)_{q})]ₛ is a [poly(D,L-Lactide)-co-poly(ethylene glycol)-co-poly(D,L-Lactide)]-b-[poly(L-lactide-co-glycolide)] multiblock copolymer wherein the L-lactide:glycolide molar ratio of R⁴ is 99:1 to 95:5 mole/mole, m is 800-1000 g/mole, n is 100-300 g/mole, p is 1800-2200 g/mole, q is 3800-4200 g/mole, and r/s is 5/95-15/85 molar ratio. In some embodiments, r/s is 5/95 to 10/90 molar ratio. In some embodiments, r/s is 10/90 to 15/85 molar ratio.

In some embodiments, the pharmaceutically acceptable salt of octreotide comprises an acetate salt, trifluoroacetate salt, or HCl salt of octreotide. In some embodiments, the pharmaceutically acceptable salt of octreotide comprises an acetate salt of octreotide. In some embodiments, the pharmaceutically acceptable salt of octreotide comprises a trifluoroacetate salt of octreotide. In some embodiments, the pharmaceutically acceptable salt of octreotide comprises a HCl salt of octreotide.

In another aspect, the dosage form further comprises a diluent. In some embodiments, the diluent comprises water, or a combination thereof. In some embodiments, the diluent comprises water. In some embodiments, the diluent comprises).

In another aspect, the dosage form further comprises an aqueous diluent.

In some embodiments, the microspheres are reconstituted in the diluent.

In some embodiments, the diluent comprises carboxymethyl cellulose aqueous solution.

In some embodiments, the microspheres are suspended in the aqueous diluent. In some embodiments, the aqueous diluent comprises carboxymethyl cellulose aqueous solution.

In some embodiments, the aqueous diluent comprises carmellose sodium, mannitol, or a combination thereof. In some embodiments, the aqueous diluent comprises carmellose sodium. In some embodiments, the aqueous diluent comprises mannitol.

In some embodiments, the dosage form further comprises an inorganic solvent.

In some embodiments, a dosage form described herein further comprises a surfactant. In some embodiments, the surfactant is dissolved in the diluent. In some embodiments, the surfactant is dispersed in the diluent. In some embodiments, the dosage form comprises about 0.01 wt % to about 20 wt % of the surfactant. In some embodiments, the dosage form comprises about 0.025 wt % to about 10 wt % of the surfactant. In some embodiments, the dosage form comprises about 0.05 wt % to about 10 wt % of the surfactant. In some embodiments, the dosage form comprises about 0.05 wt % to about 1 wt % of the surfactant. In some embodiments, the dosage form comprises about 0.05 wt % to about 5 wt % of the surfactant. In some embodiments, the dosage form comprises about 0.05 wt % to about 2 wt % of the surfactant. In some embodiments, the dosage form comprises about 0.1 wt % to about 2 wt % of a surfactant. In some embodiments, the dosage form comprises about 0.5 wt % to about 15 wt % of a surfactant. In some embodiments, the dosage form comprises about 1.0 wt % to about 10 wt % of a surfactant. In some embodiments, the dosage form comprises about 1.5 wt % to about 10 wt % of a surfactant. In some embodiments, the dosage form comprises about 2.0 wt % to about 10 wt % of a surfactant. In some embodiments, the dosage form comprises about 5.0 wt % to about 10 wt % of a surfactant. In some embodiments, the dosage form comprises about 1.0 wt % to about 9.0 wt % of a surfactant. In some embodiments, the dosage form comprises about 1.0 wt % to about 5.0 wt % of a surfactant. In some embodiments, the wt % of the surfactant is based on the weight of the diluent. In some embodiments, the wt % of the surfactant is based on the weight of the dosage form.

In some embodiments, the dosage form comprises about 0.010 wt % of a surfactant. In some embodiments, the dosage form comprises about 0,025 wt %; of a surfactant. In some embodiments, the dosage form comprises about 0.05 wt % of a surfactant. In some embodiments, the dosage form comprises about 0.1 wt % of a surfactant. In some embodiments, the dosage form comprises about 0.5 wt % of a surfactant. In some embodiments, the dosage form comprises about 1.0 wt % of a surfactant. In some embodiments, the dosage form comprises about 1.5 w % of a surfactant. In some embodiments, the dosage form comprises about 2.0 wt % of a surfactant. In some embodiments, the dosage form comprises about 2.5 wt % of a surfactant In some embodiments, the dosage form comprises about 3.0 wt % of a surfactant. In some embodiments, the dosage form comprises about 3.5 wt % of a surfactant. In some embodiments, the dosage form comprises about 4.0 wt % of a surfactant. In some embodiments, the dosage form comprises about 4.5 wt % of a surfactant In some embodiments, the dosage form comprises about 5.0 wt % of a surfactant. In some embodiments, the dosage form comprises about 6.0 wt % of a surfactant. In some embodiments, the dosage form comprises about 7.0 w % of a surfactant. In some embodiments, the dosage form comprises about 8.0 wt % of a surfactant. In some embodiments, the dosage form comprises about 9.0 wt % of a surfactant In some embodiments, the dosage form comprises about 10 wt % of a surfactant. In some embodiments, the wt % of the surfactant is based on the weight of the diluent In some embodiments, the wt % of the surfactant is based on the weight of the dosage form.

In some embodiments, a dosage form described herein comprises a surfactant, e.g., non-ionic surfactant, cationic surfactant, or anionic surfactant. In some embodiments, the surfactant comprises polysorbates, poloxamers, bile salts, glyceryl monostearate, sodium lauryl sulfate, sorbitan monooleate, polyoxyethylene sorbitan monooleate, copolymers of ethylene oxide and propylene oxide, d-α-tocopheryl polyethylene glycol succinate (Vitamin E TPGS), or a combination thereof. In some embodiments, the surfactant is selected from polysorbates, poloxamers, and a combination thereof. In some embodiments, the surfactant comprises a polysorbate (e.g., polysorbate 80). In some embodiments, the surfactant comprises a poloxamer. In some embodiments, the surfactant comprises a bile salt. In some embodiments, the surfactant comprises a glyceryl monostearate. In some embodiments, the surfactant comprises sodium lauryl sulfate. In some embodiments, the surfactant comprises sorbitan monooleate. In some embodiments, the surfactant comprises polyoxyethylene sorbitan monooleate. In some embodiments, the surfactant comprises a copolymer of ethylene oxide and propylene oxide. In some embodiments, the surfactant comprises d-α-tocopheryl polyethylene glycol succinate (Vitamin E TPGS).

In some embodiments, within the first 20 days after being administered to a mammal, the microsphere releases from about 5 wt % to about 50 wt % of the octreotide, or the pharmaceutically acceptable salt thereof, present in the microsphere. In some embodiments, within the first 20 days after being administered to a mammal, the microsphere releases from about 10 wt % to about 50 wt % of the octreotide, or the pharmaceutically acceptable salt thereof, present in the microsphere. In some embodiments, within the first 20 days after being administered to a mammal, the microsphere releases from about 15 wt % to about 50 wt % of the octreotide, or the pharmaceutically acceptable salt thereof, present in the microsphere. In some embodiments, within the first 20 days after being administered to a mammal, the microsphere releases from about 20 wt % to about 50 wt % of the octreotide, or the pharmaceutically acceptable salt thereof, present in the microsphere. In some embodiments, within the first 20 days after being administered to a mammal, the microsphere releases from about 25 wt % to about 50 wt % of the octreotide, or the pharmaceutically acceptable salt thereof, present in the microsphere. In some embodiments, within the first 20 days after being administered to a mammal, the microsphere releases from about 25 wt % to about 40 wt % of the octreotide, or the pharmaceutically acceptable salt thereof, present in the microsphere. In some embodiments, within the first 20 days after being administered to a mammal, the microsphere releases from about 30 wt % to about 40 wt % of the octreotide, or the pharmaceutically acceptable salt thereof, present in the microsphere. In some embodiments, within the first 20 days after being administered to a mammal, the microsphere releases from about 20 wt % to about 40 wt % of the octreotide, or the pharmaceutically acceptable salt thereof, present in the microsphere.

In some embodiments, within the first 60 days after being administered to a mammal, the microsphere releases from about 30 wt % to about 90 wt % of the octreotide, or the pharmaceutically acceptable salt thereof, present in the microsphere. In some embodiments, within the first 60 days after being administered to a mammal, the microsphere releases from about 40 wt % to about 90 wt % of the octreotide, or the pharmaceutically acceptable salt thereof, present in the microsphere. In some embodiments, within the first 60 days after being administered to a mammal, the microsphere releases from about 50 wt % to about 90 wt % of the octreotide, or the pharmaceutically acceptable salt thereof, present in the microsphere. In some embodiments, within the first 60 days after being administered to a mammal, the microsphere releases from about 60 wt % to about 90 wt % of the octreotide, or the pharmaceutically acceptable salt thereof, present in the microsphere. In some embodiments, within the first 60 days after being administered to a mammal, the microsphere releases from about 70 wt % to about 90 wt % of the octreotide, or the pharmaceutically acceptable salt thereof, present in the microsphere. In some embodiments, within the first 60 days after being administered to a mammal, the microsphere releases from about 70 wt % to about 85 wt % of the octreotide, or the pharmaceutically acceptable salt thereof, present in the microsphere. In some embodiments, within the first 60 days after being administered to a mammal, the microsphere releases from about 70 wt % to about 80 wt % of the octreotide, or the pharmaceutically acceptable salt thereof, present in the microsphere.

In some embodiments, within the first 20 days after being administered to a mammal, the microsphere releases at most about 10 wt % of the octreotide, or the pharmaceutically acceptable salt thereof, present in the microsphere. In some embodiments, within the first 20 days after being administered to a mammal, the microsphere releases at most about 9 wt % of the octreotide, or the pharmaceutically acceptable salt thereof, present in the microsphere. In some embodiments, within the first 20 days after being administered to a mammal, the microsphere releases at most about 8 wt % of the octreotide, or the pharmaceutically acceptable salt thereof, present in the microsphere. In some embodiments, within the first 20 days after being administered to a mammal, the microsphere releases at most about 7 wt % of the octreotide, or the pharmaceutically acceptable salt thereof, present in the microsphere. In some embodiments, within the first 20 days after being administered to a mammal, the microsphere releases at most about 6 wt % of the octreotide, or the pharmaceutically acceptable salt thereof, present in the microsphere In some embodiments, within the first 20 days after being administered to a mammal, the microsphere releases at most about 5 wt % of the octreotide, or the pharmaceutically acceptable salt thereof, present in the microsphere. In some embodiments, within the first 20 days after being administered to a mammal, the microsphere releases at most about 4 wt % of the octreotide, or the pharmaceutically acceptable salt thereof, present in the microsphere.

In some embodiments, within the first 60 days after being administered to a mammal, the microsphere releases at most about 50 wt % of the octreotide, or the pharmaceutically acceptable salt thereof present in the microsphere. In some embodiments, within the first 60 days after being administered to a mammal, the microsphere releases at most about 40 wt % of the octreotide, or the pharmaceutically acceptable salt thereof present in the microsphere.

In some embodiments, within the first 60 days after being administered to a mammal, the microsphere releases at most about 30 wt % of the octreotide, or the pharmaceutically acceptable salt thereof present in the microsphere. In some embodiments, within the first 60 days after being administered to a mammal, the microsphere releases at most about 20 wt % of the octreotide, or the pharmaceutically acceptable salt thereof present in the microsphere. In some embodiments, within the first 60 days after being administered to a mammal, the microsphere releases at most about 10 wt % of the octreotide, or the pharmaceutically acceptable salt thereof present in die microsphere

In some embodiments, the in vitro release is determined by a HPLC or UPLC method.

In some embodiments, the in vitro release is determined by an Bicinchoninic acid (BCA) assay,

In some embodiments, the in vitro release is determined by an LC/MS method.

In some embodiments, a microsphere described herein has a D50 value of 10-250 μm. In some embodiments, the microsphere has a D50 value of 10-100 μm. In some embodiments, the microsphere has a D50 value of 10-50 μm. In some embodiments, the microsphere has a D50 value of 25-75 μm. In some embodiments, the microsphere has a D50 value of 50-100 μm. In some embodiments, the microsphere has a D50 value of 30-60 μm. In some embodiments, the microsphere has a D50 value of 25-50 μm.

### Kits of Formulation

In another aspect, disclosed herein is a kit of formulation comprising: (a) a first container (e.g., a vial) comprising a microsphere, wherein the microsphere comprises octreotide, or a pharmaceutically acceptable salt thereof and a biodegradable polymer; and (b) a second container comprising a pharmaceutically acceptable diluent. In one aspect, disclosed herein is a kit of formulation comprising: (a) a first container (e.g., a vial) comprising a biodegradable polymer microsphere, wherein the biodegradable polymer microsphere comprises octreotide, or a pharmaceutically acceptable salt thereof, and a biodegradable polymer; and (b) a second container comprising a pharmaceutically acceptable diluent. In some embodiments, the biodegradable polymer microsphere comprises about 10 wt % to about 99 wt % of the biodegradable polymer. In some embodiments, the biodegradable polymer is a multiblock copolymer comprising at least one hydrolysable pre-polymer (A) segment and at least one hydrolysable pore-polymer (B) segment. In some embodiments, the segments are linked by a multifunctional chain extender. In some embodiments, the octreotide, or the pharmaceutically acceptable salt thereof, is present in the microsphere. In some embodiments, the kit further comprises one or two syringes for withdrawing the content of the first and/or the second containers. In some embodiments, the kit further comprises instructions of mixing the microsphere with the pharmaceutically acceptable diluent.

In one aspect, disclosed herein is a kit of formulation comprising: (a) a first container (e.g., a vial) comprising a biodegradable polymer microsphere, wherein the biodegradable polymer microsphere comprises octreotide, or a pharmaceutically acceptable salt thereof, and a biodegradable polymer, the biodegradable polymer is a multiblock copolymer, wherein the biodegradable multiblock copolymer comprises a [poly(D,L-Lactide-co-poly(ethylene glycol)-co-poly(D,L-Lactide)]-b-[poly(L-lactide-co-glycolide)] multiblock copolymer, wherein the biodegradable multiblock copolymer is represented by [(R¹ₘ R²ₙ R³ₘ)ₚ]ᵣ[(R⁴)_{q})]ₛ, wherein: R¹ and R³ are each a poly(D,L-Lactide) segment; R² is a poly(ethylene glycol) segment; R⁴ is a poly(L-lactide-co-glycolide) segment, wherein the L-lactide:glycolide molar ratio is 99:1 to 80:20 mole/mole; m is a molecular weight of R¹ and R³, and m is 400 to 1000 g/mole; n is a molecular weight of R², and n is 100 to 1200 g/mole; p is a molecular weight of the (R¹ₘ R²ₙ R³ₘ) segment, and p is 1800 to 2200 g/mole; q is a molecular weight of the (R⁴) segment, and q is 3800 to 4200 g/mole; and r/s is a ratio of the (R¹ₘ R²ₙ R³ₘ) segment over the (R⁴), and r/s is 5/95 to 15/85 molar ratio; and (b) a second container comprising a pharmaceutically acceptable diluent.

In some embodiments, the microsphere is in a powder form. In some embodiments, the microsphere is in a lyophilized powder form. In some embodiments, the microsphere is in a powder form which is dried by lyophilization. In some embodiments, the microsphere is in a vacuum freeze-dried powder form. In some embodiments, the microsphere is in a powder form which is dried by vacuum freeze drying. In some embodiments, the microsphere is in a vacuum-dried powder form. In some embodiments, the microsphere is in a powder form which is dried in vacuo.

In some embodiments, the kit comprises about 1 mg to about 1000 mg of the octreotide, or a pharmaceutically acceptable salt thereof. In some embodiments, the kit comprises about 25 mg to about 500 mg of the octreotide, or a pharmaceutically acceptable salt thereof. In some embodiments, the kit comprises about 50 mg to about 250 mg of the octreotide, or a pharmaceutically acceptable salt thereof. In some embodiments, the kit comprises about 75 mg to about 150 mg of the octreotide, or a pharmaceutically acceptable salt thereof. In some embodiments, the kit comprises about 100 mg to about 500 mg of the octreotide, or a pharmaceutically acceptable salt thereof. In some embodiments, the kit comprises about 100 mg to about 200 mg of the octreotide, or a pharmaceutically acceptable salt thereof. In some embodiments, the kit comprises about 50 mg to about 250 mg of the octreotide, or a pharmaceutically acceptable salt thereof.

In some embodiments, the pharmaceutically acceptable diluent is an aqueous diluent. In some embodiments, the aqueous diluent comprises Water for Injection (WFI). In some embodiments, the aqueous diluent comprises carboxymethyl cellulose aqueous solution. In some embodiments, the aqueous diluent comprises carmellose sodium, mannitol, or a combination thereof. In some embodiments, the aqueous diluent further comprises carmellose sodium. In some embodiments, the aqueous diluent further comprises mannitol.

In some embodiments, the pharmaceutically acceptable diluent further comprises a surfactant. In some embodiments, the pharmaceutically acceptable diluent further comprises about 0.01 wt % to about 20 wt % of a surfactant. In some embodiments, the pharmaceutically acceptable diluent further comprises about 0.025 wt % to about 10 wt % of a surfactant. In some embodiments, the pharmaceutically acceptable diluent further comprises about 0.025 wt % to about 5 wt % of a surfactant. In some embodiments, the pharmaceutically acceptable diluent further comprises about 0.05 wt % to about 2 wt % of a surfactant. In some embodiments, the pharmaceutically acceptable diluent further comprises about 0.01 wt % to about 5 wt % of a surfactant. In some embodiments, the pharmaceutically acceptable diluent comprises about 0.001 wt % to about 1 wt % of a surfactant. In some embodiments, the pharmaceutically acceptable diluent comprises about 0.001 wt % to about 0.5 wt % of a surfactant. In some embodiments, the pharmaceutically acceptable diluent comprises about 1.0 wt % to about 10 wt % of a surfactant. In some embodiments, the pharmaceutically acceptable diluent comprises about 1.5 wt % to about 10 wt % of a surfactant. In some embodiments, the pharmaceutically acceptable diluent comprises about 2.0 wt % to about 10 wt % of a surfactant. In some embodiments, the pharmaceutically acceptable diluent comprises about 1.0 wt % to about 9.0 wt % of a surfactant. In some embodiments, the pharmaceutically acceptable diluent comprises about 1.0 wt % to about 6.0 wt % of a surfactant. In some embodiments, the pharmaceutically acceptable diluent comprises about 1.0 wt % to about 5.0 wt % of a surfactant.

In some embodiments, the pharmaceutically acceptable diluent comprises about 0.010 wt % of a surfactant. In some embodiments, the pharmaceutically acceptable diluent comprises about 0 025 wt % of a surfactant. In some embodiments, the pharmaceutically acceptable diluent comprises about 0.05 wt % of a surfactant. In some embodiments, the pharmaceutically acceptable diluent comprises about 0.1 wt % of a surfactant disclosed herein. In some embodiments, the pharmaceutically acceptable diluent comprises about 0.5 wt % of a surfactant disclosed herein. In some embodiments, the pharmaceutically acceptable diluent comprises about 1.0 wt % of a surfactant disclosed herein. In some embodiments, the pharmaceutically acceptable diluent comprises about 1.5 wt % of a surfactant disclosed herein. In some embodiments, the pharmaceutically acceptable diluent comprises about 2.0 wt % of a surfactant disclosed herein. In some embodiments, the pharmaceutically acceptable diluent comprises about 2.5 wt % of a surfactant disclosed herein. In some embodiments, the pharmaceutically acceptable diluent comprises about 3.0 wt % of a surfactant disclosed herein In some embodiments, the pharmaceutically acceptable diluent comprises about 3.5 wt % of a surfactant disclosed herein. In some embodiments, the pharmaceutically acceptable diluent comprises about 4.0 wt % of a surfactant disclosed herein. In some embodiments, the pharmaceutically, acceptable diluent comprises about 4 5 wt % of a surfactant disclosed herein. In some embodiments, the pharmaceutically acceptable diluent comprises about 5.0 wt % of a surfactant.

In some aspects, the kit of formulation disclosed herein further comprises an instruction for reconstitution.

### Methods

In another aspect, disclosed herein is a method of making the extended release octreotide dosage form disclosed herein, comprising mixing the first vial and the second vial of the kit of formulation disclosed herein.

In another aspect, disclosed herein is a method of preventing or reducing an age-related decline in quality of life in a mammal, comprising administering to the mammal a therapeutically effective amount of the extended release octreotide dosage form disclosed herein, or providing the kit of formulation disclosed herein to the mammal.

In another aspect, disclosed herein is a method of preventing or reducing an age-related increase in frailty in a mammal, comprising administering to the mammal a therapeutically effective amount of the extended release octreotide dosage form disclosed herein, or providing the kit of formulation disclosed herein to the mammal.

In another aspect, disclosed herein is a method of improving cognitive function in a mammal in need thereof, comprising administering to the mammal a therapeutically effective amount of the extended release octreotide dosage form disclosed herein, or providing the kit of formulation disclosed herein to the mammal.

In another aspect, disclosed herein is a method of increasing lifespan, promoting longevity, and/or preventing, reducing the severity of or delaying the onset of various aging-associated conditions in a mammal in need thereof, comprising administering to the mammal a therapeutically effective amount of the extended release octreotide dosage form disclosed herein, or providing the kit of formulation disclosed herein to the mammal.

In some embodiments, increasing lifespan comprises an at least 5%, at least 10%, at least 15%, or at least 20% increase in lifespan relative to the expected or median lifespan of a mammal of similar species, strain, or breed. In some embodiments, increasing lifespan comprises an at least 5% increase in lifespan relative to the expected or median lifespan of a mammal of similar species, strain, or breed. In some embodiments, increasing lifespan comprises an at least 10% increase in lifespan relative to the expected or median lifespan of a mammal of similar species, strain, or breed. In some embodiments, increasing lifespan comprises an at least 15% increase in lifespan relative to the expected or median lifespan of a mammal of similar species, strain, or breed. In some embodiments, increasing lifespan comprises an at least 20% increase in lifespan relative to the expected or median lifespan of a mammal of similar species, strain, or breed.

In some embodiments, increasing lifespan comprises an at least 5%, at least 10%, at least 15%, at least 20%, or at least 25% increase in lifespan. In some embodiments, increasing lifespan comprises an at least 5% increase in lifespan. In some embodiments, increasing lifespan comprises an at least 10% increase in lifespan. In some embodiments, increasing lifespan comprises an at least 15% increase in lifespan. In some embodiments, increasing lifespan comprises an at least 20% increase in lifespan. In some embodiments, increasing lifespan comprises an at least 25% increase in lifespan.

In some embodiments, a therapeutically effective amount of the extended release octreotide dosage form disclosed herein inhibits production and/or release of growth hormone.

In some embodiments, a therapeutically effective amount of the extended release octreotide dosage form disclosed herein inhibits growth hormone receptor activity.

In some embodiments, a therapeutically effective amount of the extended release octreotide dosage form disclosed herein produces a decrease in the amount of activity along the growth hormone-growth hormone receptor axis.

In some embodiments, the mammal has reached maturity.

In some embodiments, the mammal has reached old age.

In some embodiments, the mammal is dog, cat, horse, cow, pig, rabbit, rodent, sheep, non-human primate, or human. In some embodiments, the mammal is dog. In some embodiments, the mammal is cat. In some embodiments, the mammal is horse. In some embodiments, the mammal is cow. In some embodiments, the mammal is pig. In some embodiments, the mammal is rabbit.

In some embodiments, the mammal is rodent. In some embodiments, the rodent is a mouse or a rat. In some embodiments, the rodent is a mouse. In some embodiments, the rodent is a rat. In some embodiments, the mammal is sheep. In some embodiments, the mammal is non-human primate. In some embodiments, the mammal is human.

In some embodiments of the extended release octreotide dosage forms, the kits of formulation, and the methods described herein, the mammal is a dog, cat, horse, cow, pig, rabbit, rodent, sheep, non-human primate, or human. In some embodiments of the extended release octreotide dosage forms, the kits of formulation, and the methods described herein, the mammal is a non-rodent. In some embodiments of the extended release octreotide dosage forms, the kits of formulation, and the methods described herein, the mammal is a dog. In some embodiments of the extended release octreotide dosage forms, the kits of formulation, and the methods described herein, the mammal is a human. In some embodiments of the extended release octreotide dosage forms, the kits of formulation, and the methods described herein, the mammal is a cat. In some embodiments of the extended release octreotide dosage forms, the kits of formulation, and the methods described herein, the mammal is a horse. In some embodiments of the extended release octreotide dosage forms, the kits of formulation, and the methods described herein, the mammal is a cow. In some embodiments of the extended release octreotide dosage forms, the kits of formulation, and the methods described herein, the mammal is a pig. In some embodiments of the extended release octreotide dosage forms, the kits of formulation, and the methods described herein, the mammal is a rabbit. In some embodiments of the extended release octreotide dosage forms, the kits of formulation, and the methods described herein, the mammal is a rodent. In some embodiments of the extended release octreotide dosage forms, the kits of formulation, and the methods described herein, the rodent is a mouse or a rat. In some embodiments of the extended release octreotide dosage forms, the kits of formulation, and the methods described herein, the mammal is a sheep. In some embodiments of the extended release octreotide dosage forms, the kits of formulation, and the methods described herein, the mammal is a non-human primate.

In some embodiments of the extended release octreotide dosage forms, the kits of formulation, and the methods described herein, the mammal has reached maturity. As used herein, the term mature or maturity, and the like, refers to a mammal that is capable of sexual reproduction and/or a mammal that has achieved its adult height and/or length.

In some embodiments of the extended release octreotide dosage forms, the kits of formulation, and the methods described herein, the mammal is administered a herein disclosed the extended release octreotide dosage form as it is nearing or once it has reached halfway to its expected lifespan for the mammal's species, strain, breed, sex, and/or age. The mammal may have reached an age that is at least 60%, 70%, 80%, 90%, or 100% of its expected lifespan for the mammal's species, strain, breed, sex, and/or age.

It is known that small dog breeds (e.g., Chihuahua) have longer expected lifespans than larger dog breeds (e.g., Great Dane). Accordingly, a Chihuahua, which has an expected lifespan of 15 years, will reach halfway to its expected lifespan at about 7 years (or earlier); thus, a Chihuahua may be administered a pharmaceutical formulation disclosed herein beginning around 7 years of age. On the other hand, a Great Dane, which has an expected lifespan of 7 years, will halfway to its expected lifespan at about 3 years; thus, a Great Dane may be administered a pharmaceutical formulation disclosed herein beginning around 3 years of age (or earlier). As disclosed herein, a mammal may be administered the extended release octreotide dosage forms described herein once it has reached maturity; thus, either dog breed may be administered a composition about its first-year birthday.

Any dog breed can be administered an extended release octreotide dosage form disclosed herein and treated by a herein described method. Lists of the most common dog breeds, identified by year, are maintained by the American Kennel Club. See, the World Wide Web (www) at akc.org/expert-advice/news/most-popular-dog-breeds-full-ranking-list; the lists of dog breeds, published at the time of the present application's filing, are incorporated by reference in their entireties. Illustrative common dog breeds include Retrievers (Labrador), German Shepherd Dogs, Retrievers (Golden), French Bulldogs, Bulldogs, Beagles, Poodles, Rottweilers, Pointers (German Shorthaired), Yorkshire Terriers, Boxers, Dachshunds, Pembroke Welsh Corgis, Siberian Huskies, Australian Shepherds, Great Danes, Doberman Pinschers, Cavalier King Charles Spaniels, Miniature Schnauzers, Shih Tzu, Boston Terriers, Bernese Mountain Dogs, Pomeranians, Havanese, Shetland Sheepdogs, Brittanys, Spaniels (English Springer), Pugs, Mastiffs, Spaniels (Cocker), Vizslas, Cane Corso, Chihuahuas, Miniature American Shepherds, Border Collies, Weimaraners, Maltese, Collies, Basset Hounds, and Newfoundlands. In some embodiments, the dog can be cross-breed, mixed-breed, or a dog of unknown breed. In some embodiments, the dog is cross-breed or mixed-breed of above.

In some embodiments of the extended release octreotide dosage forms, the kits of formulation, and the methods described herein, the human is an adult human. In some embodiments, the human has an age in a range of from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old, or older.

In some embodiments, the mammal is a dog of any age. In some embodiments, the mammal is a dog over the age of 7. In some embodiments, the mammal is a dog of 1-3 years old. In some embodiments, the mammal is a dog of 1-3 years old. In some embodiments, the mammal is a dog of 4-8 years old. In some embodiments, the mammal is a dog of at least 7 years old. In some embodiments, the mammal is a dog of at least 9 years old. In some embodiments, the mammal is a dog of at least 10 years old. In some embodiments, the mammal is a dog of at least 11 years old. In some embodiments, the mammal is a dog of at least 12 years old. In some embodiments, the mammal is a dog of at least 13 years old. In some embodiments, the mammal is a dog of at least 14 years old. In some embodiments, the mammal is a dog of at least 15 years old. In some embodiments, the mammal is a dog with signs of aging. In some embodiments, the mammal is a dog that can benefit from a stable glucose level. In some embodiments, the mammal is a dog that is susceptible to a change of glucose level. In some embodiments, the mammal is a dog that is susceptible to glucose elevation. In some embodiments, the mammal is a dog with signs of aging. In some embodiments, the mammal is a dog that can benefit from a lower saturated fatty acid level in the body. In some embodiments, the mammal is a dog that has high saturated fatty acid level in the body. In some embodiments, the mammal is a dog that has high palmitic acid level in the body.

In some embodiments of the extended release octreotide dosage forms, the kits of formulation, and the methods described herein, the mammal is a non-human animal, and therefore the invention pertains to veterinary use. In a specific embodiment, the non-human animal is a household pet or a service animal, e.g., a dog. In another specific embodiment, the non-human animal is a livestock animal.

The extended release octreotide dosage forms, the kits of formulation, and the methods described herein treat, prevent, reduce the severity of, and/or delay the onset of various aging-associated conditions, e.g., cellular senescence, chronic diseases and disabilities/conditions of aging. Illustrative aging-associated conditions include age-related macular degeneration (AID), Alzheimer's disease, arthritis, atherosclerosis and cardiovascular disease, benign prostatic hyperplasia (BPH), bone atrophy, cancer, cardiovascular decline, cataracts, constipation, decrease in visual acuity, decrease in overall energy, delirium, dementia, depression, diminished peripheral vision, greater risk of heat stroke or hypothermia, hearing loss, hypertension, increased inflammation, increased susceptibility to infection (including influenza and pneumonia), kidney disease, memory loss, metabolic syndrome, muscle atrophy, osteoporosis, reduced metabolism (including increased risk for obesity), reduced reflexes and coordination including difficulty with balance, respiratory disease, shingles, type 2 diabetes, urologic changes (including incontinence), whitening or graying of hair, and wrinkling and sagging skin (including loss of skin elasticity). Aged non-human subjects experience similar, homologous, and/or equivalent aging-associated conditions.

### Definitions

The singular forms “a,” “an,” and “the” include plural referents unless the context clearly dictates otherwise. Thus, for example, reference to “a drug” includes reference to one or more of such drugs, and reference to “an excipient” includes reference to one or more of such excipients. When ranges are used herein, all combinations and sub-combinations of ranges and specific embodiments therein are intended to be included.

The term “about” when referring to a number or a numerical range means that the number or numerical range referred to is an approximation within experimental variability (or within statistical experimental error), and thus the number or numerical range varies no more than 15% of the stated number or numerical range.

The terms “formulation” and “composition,” as used herein, are used interchangeably and refer to a mixture of two or more compounds, elements, or molecules. In some aspects, the terms “formulation” and “composition” may be used to refer to a mixture of one or more active agents with a carrier or other excipients.

The terms “active agent,” “active pharmaceutical agent,” “drug,” “active ingredient,” and variants thereof are used interchangeably to refer to an agent or substance that has measurable specified or selected physiologic activity when administered to a subject in a significant or effective amount.

The term “pharmaceutically acceptable salt” in reference to octreotide refers to a salt of octreotide, which does not cause significant irritation to a mammal to which it is administered and does not substantially abrogate the biological activity and properties of the compound. A wide variety of pharmaceutically acceptable salts are formed from octreotide and include:
acid addition salts formed by reacting octreotide with an organic acid, which includes aliphatic mono- and dicarboxylic acids, phenyl-substituted alkanoic acids, hydroxyl alkanoic acids, alkanedioic acids, aromatic acids, aliphatic and aromatic sulfonic acids, amino acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, benzenesulfonic acid, p-toluenesulfonic acid, salicylic acid, and the like;
acid addition salts formed by reacting octreotide with an inorganic acid, which includes hydrochloric acid, hydrobromic acid, sulfuric acid, nitric acid, phosphoric acid, hydroiodic acid, hydrofluoric acid, phosphorous acid, and the like.

In some embodiments of the pharmaceutical formulations described herein, the octreotide is in the free base form. In some embodiments of the pharmaceutical formulations described herein, the octreotide is a salt. In some embodiments of the pharmaceutical formulations described herein, the octreotide is an acetate salt. In some embodiments of the pharmaceutical formulations described herein, the octreotide is a trifluoroacetate salt. In some embodiments of the pharmaceutical formulations described herein, the octreotide is a hydrochloride salt.

It should be understood that a reference to a pharmaceutically acceptable salt includes the solvent addition forms (solvates). Solvates contain either stoichiometric or non-stoichiometric amounts of a solvent, and are formed during the process of product formation or isolation with pharmaceutically acceptable solvents such as water, ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In one aspect, solvates are formed using, but not limited to, Class 3 solvent(s). Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005). Hydrates are formed when the solvent is water, or alcoholates are formed when the solvent is alcohol. In some embodiments, solvates of octreotide, or pharmaceutically acceptable salts thereof, are conveniently prepared or formed during the processes described herein. In some embodiments, solvates of octreotide are anhydrous. In some embodiments, octreotide, or pharmaceutically acceptable salts thereof, exist in unsolvated form. In some embodiments, octreotide, or pharmaceutically acceptable salts thereof, exist in unsolvated form and are anhydrous.

In yet other embodiments, octreotide, or a pharmaceutically acceptable salt thereof, is prepared in various forms, including but not limited to, amorphous phase, crystalline forms, milled forms and nano-particulate forms. In some embodiments, octreotide, or a pharmaceutically acceptable salt thereof, is amorphous. In some embodiments, octreotide, or a pharmaceutically acceptable salt thereof, is amorphous and anhydrous. In some embodiments, octreotide, or a pharmaceutically acceptable salt thereof, is crystalline. In some embodiments, octreotide, or a pharmaceutically acceptable salt thereof, is crystalline and anhydrous.

While not intending to be bound by any particular theory, certain solid forms are characterized by physical properties (e.g., stability, solubility and dissolution rate) appropriate for pharmaceutical and therapeutic dosage forms. Moreover, while not wishing to be bound by any particular theory, certain solid forms are characterized by physical properties (e.g., density, compressibility, hardness, morphology, cleavage, stickiness, solubility, water uptake, electrical properties, thermal behavior, solid-state reactivity, physical stability, and chemical stability) affecting particular processes (e.g., yield, filtration, washing, drying, milling, mixing, tableting, flowability, dissolution, formulation, and lyophilization) which make certain solid forms suitable for the manufacture of a solid dosage form. Such properties can be determined using particular analytical chemical techniques, including solid-state analytical techniques (e.g., X-ray diffraction, microscopy, spectroscopy and thermal analysis), as described herein and known in the art.

The terms “effective amount” or “therapeutically effective amount” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case may be determined using techniques, such as a dose escalation study.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

In one aspect, the present disclosure relates to a biodegradable polymer microsphere. The synthesis of the biodegradable polymer neurosphere is generally described in the U.S. patent application Ser. No. 17/773,520 (esp., Example 2), filed Oct. 30, 2020 and published as US20220409730A1; U.S. patent application Ser. No. 17/281,902 (esp., Example 4), filed Oct. 2, 2019 and published as US20220062177A1; U.S. patent application Ser. No. 14/233,961(esp., Example 17), filed Jul. 23, 2012 and published as US20140199385A1; U.S. patent application Ser. No. 15/677,442 (esp., Example 17), filed Aug. 15, 2017 and published as US20180085318A1; and U.S. patent application Ser. No. 17/765,389 (esp., Example 11), filed Sep. 30, 2020 and published as US20220332900A1; each of which is hereby incorporated by reference in its entirety.

### Example 1: Insulin Resistance HRQL Study

An observational Healthspan study was performed which involved a health-related quality of life assessment (HRQL). The study endpoints included owner assessment of Health Related Quality of Life (HRQL) and veterinarian assessment of Physical Examination, Body Condition Score (BCS), Muscle Condition Score, Complete Blood Count, biochemical profile, serum T4 measurement, urinalysis, insulin-like growth factor-1 measurement, and Canine Frailty Index (CFI). Blood samples were collected to assess serum for fasted insulin and adiponectin levels. DNA samples were also collected. Target demographics were collected within 451 eligible adult dogs, 43.6% of which were mixed breed and 56.4% of which were pure breed. Target age and size of dogs on which enrollment was based is shown below in Table 1. Approximately 451 dogs were evaluated, and about 450 dogs were assessed using the HRQL. Approximately 409 dogs were assessed for fasted insulin levels, and approximately 352 dogs were assessed for adiponectin levels. Bivariate relationships found between insulin and age, insulin and weight, insulin and BCS, insulin and HRQL, and insulin and CFI are shown below in Table 2.

TABLE 1
Demographics of dogs enrolled in HRQL study

| Group | Number of dogs | Demographics |
|---|---|---|
| A: “young, small” | 55 | ≥2 and <6 years |
| | | of age; <25 lbs. |
| B: “old, small” | 117 | ≥7 years of |
| | | age; <25 lbs. |
| C: “young, large” | 127 | ≥2 and <6 years |
| | | of age; >50 lbs. |
| D: “old, large” | 152 | ≥7 years of |
| | | age; > 50 lbs. |
| Total | 451 | — |

TABLE 2
Correlation coefficients evaluating bivariate relationships
between factors of interest in HRQL study

| | Variable | rho | p-value |
|---|---|---|---|
| Demographics | Age (yrs) | 0.18 | <0.001 |
| | Weight (lbs) | 0.07 | 0.156 |
| | Body Condition Score | 0.27 | <0.001 |
| Health Outcomes | HRQL | −0.25 | <0.001 |
| | CFI | 0.10 | 0.034 |

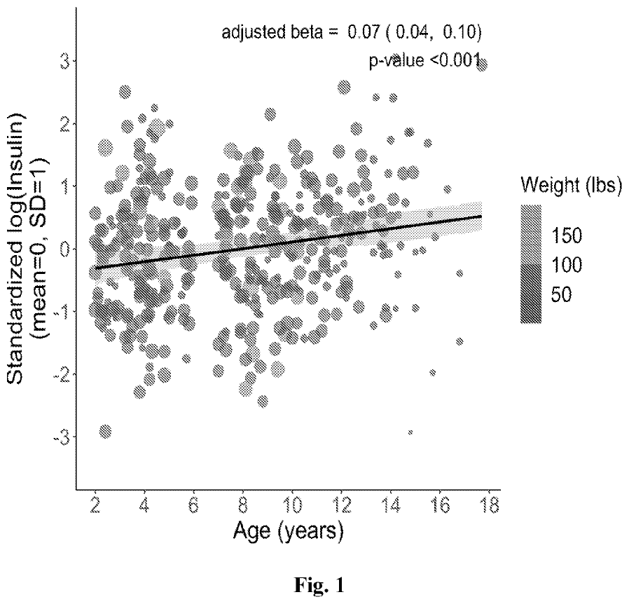

The data from the HRQL study was analyzed using a quantile regression model to estimate joint effects of the variables in Table 2. Multiple median regression was performed using a full main effects model, an interaction model with age multiplied by weight, and an interaction model with age and BCS, shown below in Table 3. A graphical representation of the association between age and standardized log(insulin) for dogs in different weight groups is shown in FIG. 1.

TABLE 3
Multiple median regression results for variables in the HRQL study

| | | Effect on standardized | | | |
|---|---|---|---|---|---|
| | | log(Insulin) | | | |
| | | Coefficient | | p- | |
| Model | Variables | (95% CI) | | value | R¹ |
| Full main | (Intercept) | −2.22 | (−2.94, −1.50) | <0.001 | 0.07 |
| effects model | Age (yrs) | 0.07 | (0.04, 0.10) | <0.001 | |
| | Weight (lbs) | 0.00 | (0.00, 0.00) | 0.573 | |
| | BCS | 0.30 | (0.17, 0.42) | <0.001 | |
| Age × weight | (Intercept) | −2.17 | (−3.04, −1.30) | <0.001 | 0.07 |
| | Age (yrs) | 0.06 | (0.00, 0.12) | 0.047 | |
| | Weight (lbs) | 0.00 | (−0.01, 0.01) | 0.986 | |
| | BCS | 0.30 | (0.17, 0.43) | <0.001 | |
| | Age × weight | 0.00 | (0.00, 0.00) | 0.775 | |
| Age × BCS | (Intercept) | −2.88 | (−4.41, −1.35) | <0.001 | 0.07 |
| | Age (yrs) | 0.15 | (−0.02, 0.32) | 0.086 | |
| | Weight (lbs) | 0.41 | (0.14, 0.69) | 0.004 | |
| | BCS | 0.00 | (0.00, 0.01) | 0.359 | |
| | Age × BCS | −0.02 | (−0.05, 0.02) | 0.345 | |

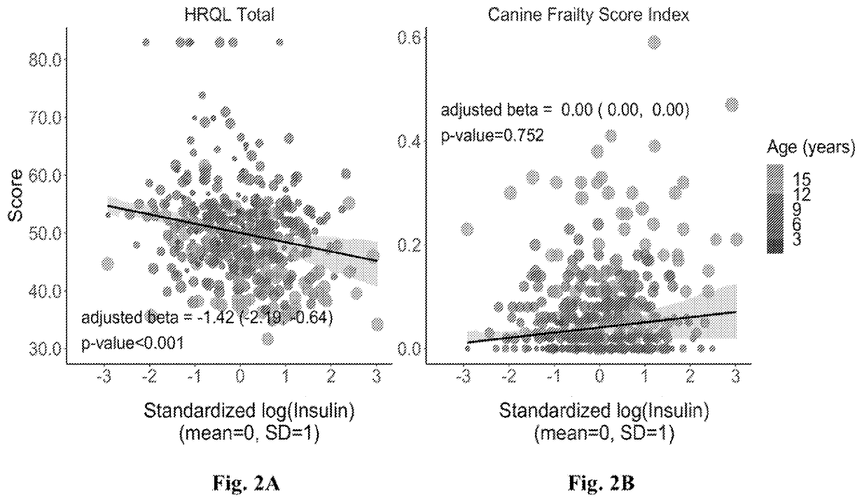

Fasted insulin levels were examined to determine their relationship with HRQL scores in the Healthspan Study. To determine HRQL, a survey was administered to owners of participants to measure owner perceptions of their dog's quality of life. The questionnaire included about 22 questions concerning the impacts of aging, disease, and clinical management of disease. These questions encompassed inquiries about the dog's behavior for areas including Energetic/Enthusiastic (E/E), Happy/Content (H/C), Active/Comfortable (A/C), and Calm/Relaxed (C/R). The answers to these questions were composited or combined into a single score known as total HRQL. The Davies transformation (Davies 2019) was applied to normalize the scores. Multiple median regression models were used to discern the relationship between insulin, HRQL, and CFI. Standardized log(insulin) was the primary variable, with age, weight, and BCS as covariates, results of which are shown below in Table 4. Interaction terms were added into models for which insulin and other covariates were significant, as shown in FIG. 2A and FIG. 2B.

TABLE 4
Multiple median regression of insulin blood levels with
HRQL and CFI adjusting for age, weight, and BCS

| Outcome | Variables | Coefficient (95% CI) | | p-value | R¹ |
|---|---|---|---|---|---|
| HRQL Total | (Intercept) | 56.66 | (51.72, 61.59) | <0.001 | 0.11 |
| | Standardized | −1.42 | (−2.19, −0.64) | <0.001 | |
| | log(Insulin) | | | | |
| | Age (yrs) | −0.76 | (−0.96, −0.56) | <0.001 | |
| | Weight (lbs) | −0.02 | (−0.04, 0.00) | 0.114 | |
| | BCS | −0.05 | (−0.91, 0.81) | 0.905 | |
| Canine Frailty | (Intercept) | 0.00 | (−0.03, 0.02) | 0.905 | 0.31 |
| Score Index | Standardized | 0.00 | (0.00, 0.00) | 0.752 | |
| | log(Insulin) | | | | |
| | Age (yrs) | 0.01 | (0.01, 0.01) | <0.001 | |
| | Weight (lbs) | 0.00 | (0.00, 0.00) | 0.387 | |
| | BCS | −0.01 | (−0.01, 0.00) | 0.003 | |

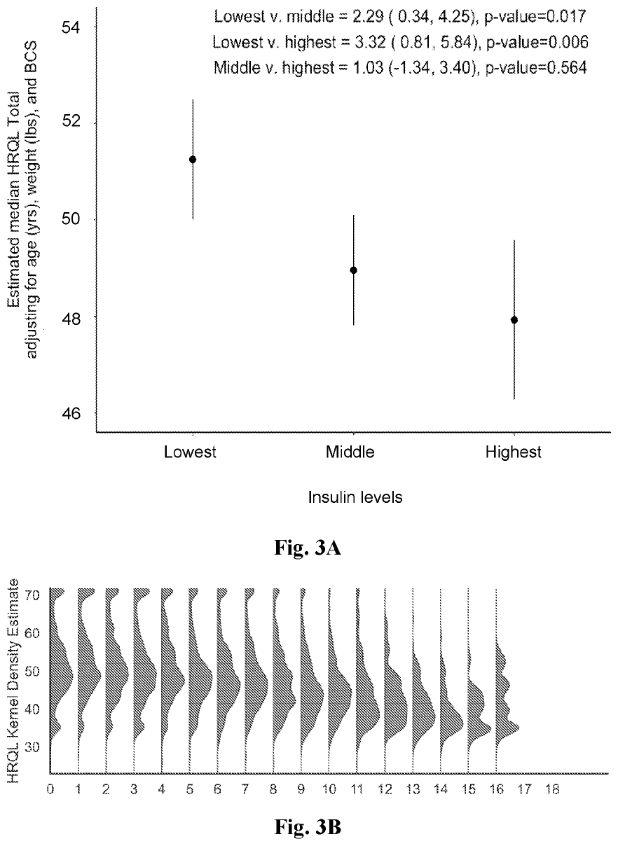

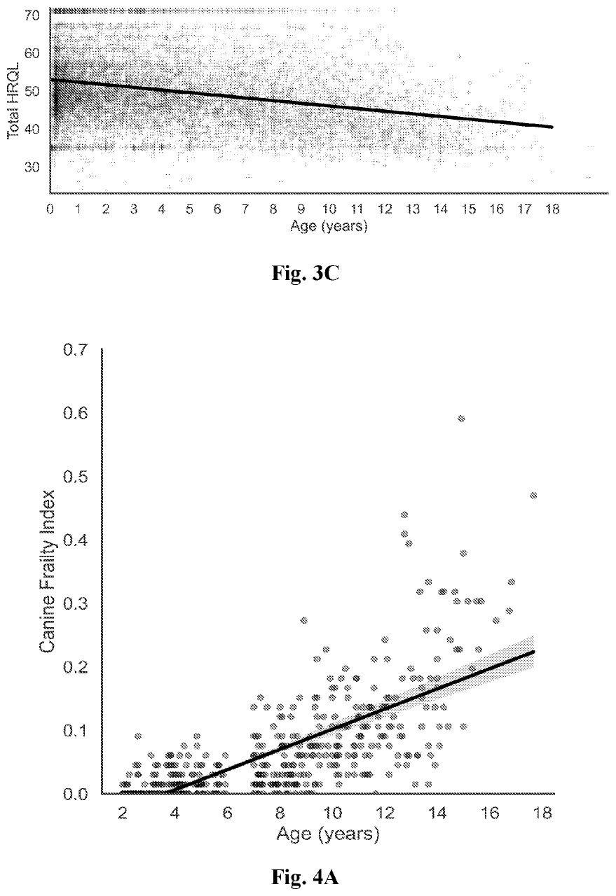

HRQL total was then compared with fasted insulin levels by categorizing the insulin levels into three groups of equal size, or tertiles. The tertiles reflected insulin levels of the lowest third, middle third, and highest third of the sample. Quantile median regression was used to estimate HRQL total based on each insulin tertile, adjusted for covariates including age, weight, and BCS. The 407 insulin measures taken along with HRQL total scores were divided into insulin tertiles, defined as: lowest insulin levels (n=137, range=[2.53 mU/L, 12 mU/L]), highest insulin (n=136, range=[20.9 mU/L, 107 mU/L]), and medium insulin levels (n=136, range=[12 mU/L, 20.9 mU/L]). The tertiles were within normal laboratory reference ranges. Multiple quantile median regression was performed for each insulin tertile group, shown in Table 5 below. The HRQL scores for each insulin tertile group, adjusting for covariates, is shown in FIG. 3A. The HRQL kernel density estimate was analyzed, shown in FIG. 3B. The HRQL total score on the Davies scale as a function of age was analyzed, shown in FIG. 3C.

TABLE 5
Multiple quantile median regression model showing
the effects of insulin tertile groups adjusting
for age, weight, and BCS as covariates (n = 407)

| | | | | | LRT¹ |
|---|---|---|---|---|---|
| | | Coefficient | | p- | p- |
| Outcome | Variables | (95% CI) | | value | value |
| HRQL | (Intercept) | 57.55 | (52.87, 62.23) | <0.001 | |
| Total | | | | | |
| | Age (yrs) | −0.76 | (−0.97, −0.55) | <0.001 | |
| | Weight (lbs) | −0.01 | (−0.04, 0.01) | 0.315 | |
| | BCS | 0.07 | (−0.76, 0.90) | 0.873 | |
| | Insulin tertile | | | | 0.005 |
| | Low: [2.53 mU/L, | referent | | | |
| | 12 mU/L]) | | | | |
| | Middle: (12 mU/L, | −2.36 | (−4.07, −0.66) | 0.007 | |
| | 20.9 mU/L] | | | | |
| | High: (20.9 mU/L, | −3.04 | (−5.10, −0.99) | 0.004 | |
| | 107 mU/L] | | | | |

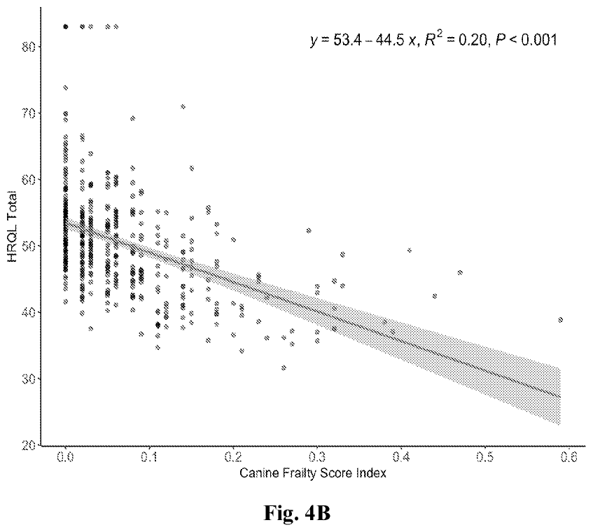

The Canine Frailty Index (CFI) scores were based on the medical history and physical examination of the dog participants, performed by a veterinarian. CFI was calculated by summing the response scores and dividing by the total number of questions. A frailty index of 0 denotes a dog that is not at all frail. Lower frailty scores indicated fewer health deficits and lower frailty, while higher frailty scores indicated more health deficits or higher frailty. The CFI score in relation to age was analyzed, shown in FIG. 4A. The CFI score in relation to total HRQL score was analyzed, as shown in FIG. 4B.

Longitudinal data can be collected to further the Healthspan study, including HRQL and mortality data for dog participants. Lipid levels can also be analyzed against HRQL score, CFI score, or both.

### Example 2: SandostatinLAR Impact on Glucose and Insulin Levels

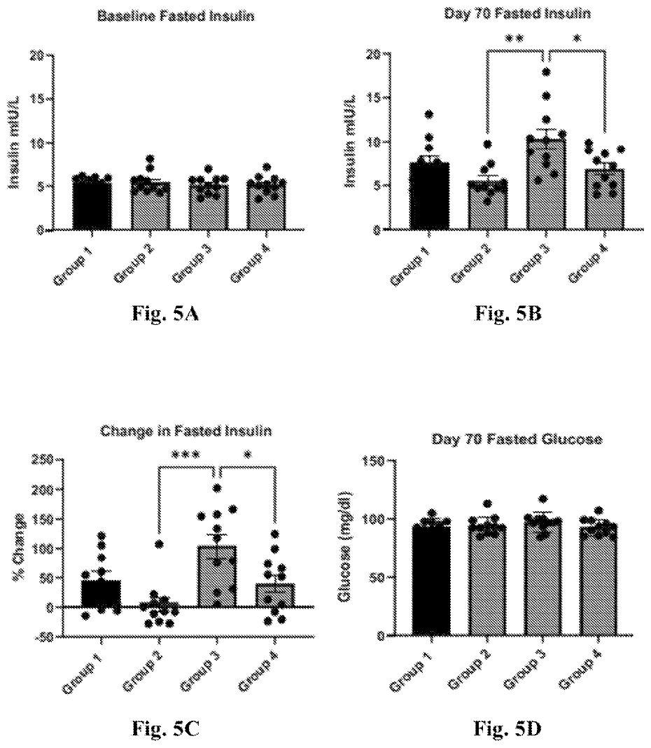

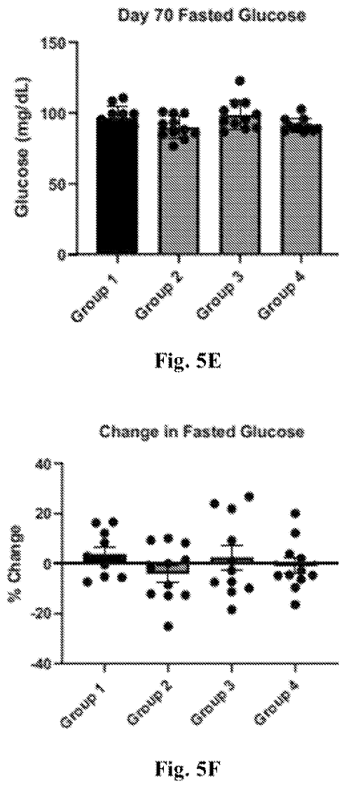

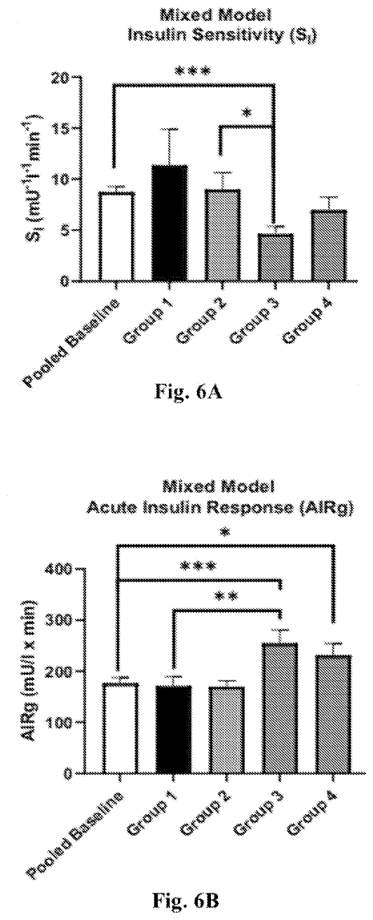

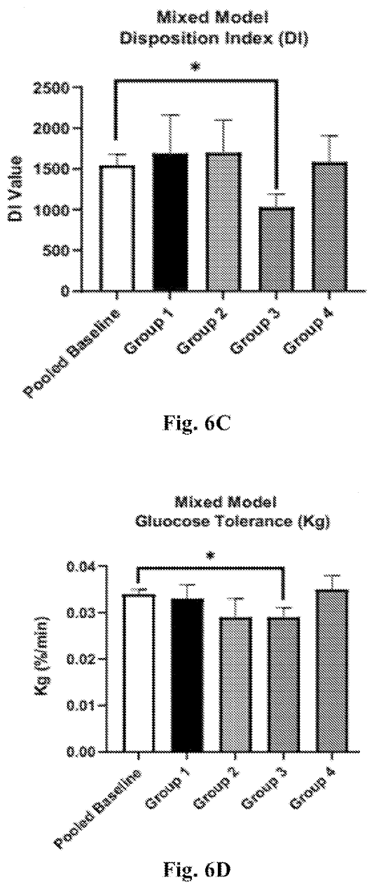

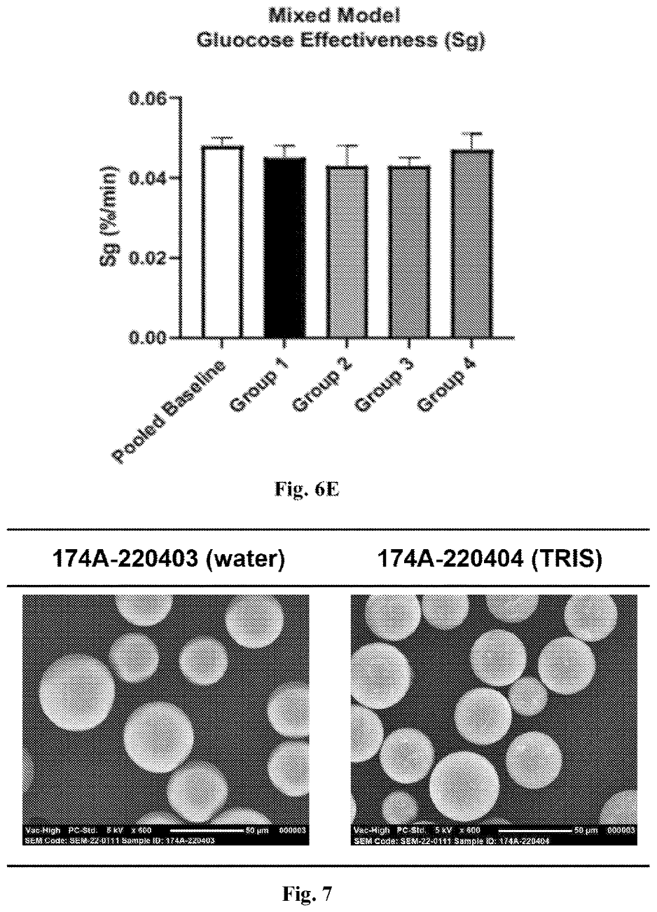

Dog participants underwent treatment with SandostatinLAR (octreotide) and were given an intravenous glucose tolerance test (IVGTT). Participants also underwent blood sampling to measure fasting insulin and whole body insulin sensitivity after treatment with SandostatinLAR. Glucose tolerance was tested after treatment with SandostatinLAR, the results of which are seen in FIGS. 5A-5F. Insulin levels were gathered and analyzed after treatment with SandostatinLAR, the results of which are in FIGS. 6A-6E. This study demonstrates that treatment with SandostatinLAR has no impact on fasting glucose measurements. This study also demonstrates that SandostatinLAR has no impact on the response of insulin to glucose or glucose effectiveness evaluated via a glucose tolerance test. This study further demonstrates that SandostatinLAR impacts fasting insulin and whole body insulin sensitivity while not impacting glucose measurements.

IVGTTs methods: IVGTTs were performed on Day 12 (pre-treatment baseline) and Day 70 (post-treatment). Briefly, IVGTTs were carried out by the following methods. Dogs were fasted overnight prior to conducting the procedure. Blood was sampled and serum and plasma separated at 10 minutes and 1 minute pre-glucose bolus to establish fasted glucose and insulin values. At 0 min (t=0), a glucose bolus (0.3 g/kg, i.v.) was delivered. At t=20 min, a bolus of insulin (0.03 U/kg, i.v.) was delivered. Blood was collected at 2, 4, 6, 10, 14, 19, 22, 30, 40, 50, 60, 80, 100, 120, 150, and 180 min post-glucose bolus. Serum was then analyzed for glucose and plasma was analyzed for insulin levels at each time point. In a blinded fashion, glucose and insulin values were entered into the MINMOD Millenium 6.02 software to generate parameter estimates for insulin sensitivity (Si), glucose effectiveness (Sg) and glucose tolerance (Kg) for each animal. Acute insulin response to glucose (AIRg) was calculated from the AUC of insulin from t=0 to t=10) and the disposition index (DI) was calculated by the multiplication of AIRg and Si. In total, it was 12 weeks of treatment.

### Example 3: Octreotide Microsphere Formulations Via W/O/W Emulsification

Octreotide-loaded microspheres (OCT-MSP) were prepared of several biodegradable multiblock copolymers using a membrane emulsification-based microencapsulation process.

[Poly(D,L-Lactide)-co-poly(ethylene glycol)-co-poly(D,L-lactide)]-b-[poly(glycolide-co-L-lactide)] multiblock copolymers with a 10/90 block ratio were synthesized using similar procedures as described in WO 2013/015685 by chain-extending poly(DL-Lactide)-co-PEG200-co-poly(DL-Lactide) or poly(DL-Lactide)-co-PEG1000-co-poly(DL-Lactide) pre-polymers with Mₙ of around 2000 g/mol and poly(glycolide-co-L-lactide) pre-polymers with Mₙ of about 4000 g/mol and a 3/97 or 15/85 glycolide/L-lactide molar ratio with 1,4-butanediisocyanate. The polymers are abbreviated as “10LP2L20-GLL40 (3/97)” and “10LP10L20-GLL40 (15/85)”. 10LP2L20-GLL40 (3/97) and 10LP10L20-GLL40 (15/85) are semi-crystalline multiblock copolymers with a glass transition of 55 and 44° C., respectively, and a melting temperature of 125 and 72° C., respectively (Table 1).

A [poly(ε-caprolactone)-co-poly(ethylene glycol)-co-poly(ε-caprolactone)]-b-[poly (p-dioxanone)] multi-block copolymer with a block ratio of 10/90 (abbreviated as “10CP6C12-D27”) was synthesized using similar procedures as described in WO 2020/071912 by chain-extending a [poly(ε-caprolactone)-co-PEG600-co-poly(6-caprolactone)] pre-polymer with a molecular weight of 1200 g/mole and a semi-crystalline poly(p-dioxanone) pre-polymer with a molecular weight of 2700 g/mole with 1,4-butanediisocyanate. 10CP6C12-D27 is a semi-crystalline multiblock copolymer with a glass transition of −15° C. and a melting temperature of 91° C. (Table 1).

Poly(glycolide-co-D,L-Lactide)-b-poly(D,L-lactide) with a 10/90 block ratio was synthesized using similar procedures as described in WO 2013/015685 by chain-extending poly(glycolide-co-DL-Lactide) pre-polymers with Mₙ of around 2000 g/mol and poly(D,L-lactide) pre-polymers with Mₙ of about 4000 g/mol with 1,4-butanediisocyanate. The polymer is abbreviated as “10GL20-L40”. 10GL20-L40 is an amorphous multiblock copolymer with a glass transition of 50° C. (Table 1).

All polymers were analysed for their chemical composition, intrinsic viscosity, residual p-dioxane content, and thermal characteristics as described in WO 2013/015685 and WO 2020/071912. Table 6 lists the characteristics of the various multiblock copolymers.

TABLE 6
Characteristics of multiblock copolymers used for the preparation
of octreotide-loaded microspheres (OCT-MSP).

| | | Block | | | | |
|---|---|---|---|---|---|---|
| | | ratio | PEG MW | IV | Tg | Tₘ |
| RCP | Polymer grade | (w/w) | (g/mol) | (dl/g) | (° C.) | (° C.) |
| 2003A | 10LP2L20-GLL40 | 10/90 | 200 | 0.76 | 55 | 125 |
| | (3/97) | | | | | |
| 2212 | 10LP10L20-GLL40 | 10/90 | 1000 | 0.83 | 44 | 72 |
| | (15/85) | | | | | |
| 1806 | 10CP6C12-D27 | 10/90 | 600 | 0.67 | −15 | 91 |
| 1927 | 10GL20-L40 | 10/90 | NA | 0.75 | 50 | NA |

A 4.0 wt. % polyvinyl alcohol (“PVA”) solution was prepared by dissolving 40 g of PVA with a viscosity of 4.3-5.7 mPa·s (40 g/L; water) and a degree of hydrolysis (USP) of 85-89% (PVA 5-88 EMPROVE® ESSENTIAL, Merck KGaA) in 960 g of ultrapure (UP)-water, followed by filtration of the solution over a 5 μm polyvinylidene difluoride (PVDF) membrane (Durapore®). 500 g of the 4 wt. % PVA solution was diluted with 4250 g UP water and 250 g of NaCl was added under stirring to obtain a 0.4 wt. % PVA solution with 5% NaCl. After complete dissolution of NaCl, the resulting solution was filtered over 0.2 μm polyethersulfone (“PES”) filter capsule (Satopore®).

Similarly, a 0.4 wt. % PVA solution with 50 mM tris(hydroxymethyl) aminomethane (“TRIS”) pH 8.3 was prepared by dissolving 7.5 gram of Trizma pre-set crystal (Sigma-Aldrich) (Mw~150.6 Da) in 1.0 L of 0.4 wt % PVA, 5 wt % NaCl solution. The resulting solution was filtered over 0.2 μm polyethersulfone (“PES”) filter capsule (Satopore®).

A 50 mM acetate buffer pH 5.5 was prepared by dissolving 0.41 g sodium acetate (Sigma-Aldrich) in 100 mL UP water at room temperature. After complete dissolution, pH of the solution was adjusted to pH 5.5 with HCl 1M.

A 0.05 wt % Tween-80 solution was prepared by adding 2.5 g of Tween-80 into 5 L of UP water and stirring the solution for 15 minutes at room temperature.

0.6 wt % aqueous carboxymethyl cellulose (“CMC”) solution (injection vehicle/diluent) was prepared by dissolving 3 g of CMC in 500 g of water for injection (“WFI”) under stirring at a temperature of 75° C. Upon dissolution of all CMC, 50 g of phosphate buffered saline (“PBS”) (10×, pH 7.4) and additional WFI was added to obtain the required concentration.

OCT-MSP with a target octreotide base (OCT) loading of 10, 15 or 20 wt. % were prepared of 10LP10L20-GLL40 (15/85), 10LP2L20-GLL40 (3/97), 10GL20-L40 and 10CP6C12-D27 at a scale of 1 g via water-in-oil-in-water (“W₁/O/W₂”) membrane emulsification followed by solvent extraction/evaporation. Octreotide acetate (“OA”) was dissolved in acetate buffer pH 5.5 at a concentration of 300 mg/mL whereafter the OA solution (W₁) was filtered via a 0.2 μm PES filter. The polymers were dissolved in dichloromethane (“DCM”) at a concentration of 10 wt. % whereafter the solution (O) was filtered over a 0.2 μm polytetrafluoroethylene (“PTFE”) filter. Subsequently, the OA solution was emulsified with the polymer solution using an Ultraturrax (21600 rpm, 40 s) and W₁/O ratios of 1:27, 1:17 or 1:12 (w/w) for target OCT loadings of 10, 15 or 20 wt. %. The so-obtained W₁/O primary emulsion (or dispersed phase, “DP”), was subsequently emulsified with an aqueous solution (W₂) of 0.4 wt. % PVA/5 wt. % NaCl solution or 0.4 wt. % PVA/5 wt. % NaCl in TRIS buffer pH 8.3 as continuous phase (“CP”) by pumping DP at a rate of 1.6 mL/min via a stainless steel membrane with 20 μm pores into a 2 L stirred vessel into which CP was pumped in parallel at a CP/DP ratio of 150 (v/v). The formed W₁/O/W₂ emulsion was stirred at 200 rpm for 3 hours at room temperature under an airflow of 5 L/min to extract and evaporate DCM and harden the microspheres. After completion of solvent evaporation, the hardened microspheres were collected by filtration (5 μm PVDF filter) and washed three times with 250 mL 0.05 wt. % Tween-80 solution and three times with 250 mL WFI. Finally, the semi-dry microspheres were transferred into 6 mL lyophilization vials, placed in a Christ freeze-dryer (Alpha 2-4 LSC plus, Germany), frozen using a shelf temperature of −45° C. followed by primary drying for 2 hours at −10° C. and 2.0 mbar and secondary drying for 15 hours at 20-30° C. with pressure decreasing from 2.0 to 0.001 mbar. Finally, the microspheres were dried at 5° C. and 0.001 mbar for 72 hours.

Microspheres were analysed for their particle size distribution (“PSD”) using a HORIBA® LA-960 Laser Particle Size Analyser.

Characterization of microsphere surface morphology and particle shape was performed by scanning electron microscopy (“SEM”) using a JEOL® JCM-5000 NEOSCOPE™.

Total content of octreotide base in OCT-MSPs was determined in duplicate by BCA Protein Assay. Briefly, 10 mg of OCT-MSP was dissolved in 1 mL of DMSO at 80° C. whereafter 5.00 mL of 0.5% SDS in 0.05 M NaOH was added. The mixture was stirred overnight on a roller mixer at room temperature to hydrolyse the polymer matrix. Then, 100 μL of sample was collected and added to 2 mL of BCA reagent followed by incubation at 50° C. for 10 minutes. The absorbance was measured by UV-VIS spectroscopy using a wavelength of 562 nm. Calibration curve from absorption of standards against absolute amount of OCT in standards was constructed using a quadratic model and a 1/X weighting factor. The OCT amount in samples was calculated by interpolation on calibration curve.

The in vitro release of OCT from OCT-MSP was determined by incubating 10 mg of OCT-MSP in 2 mL polypropylene centrifuge tube containing 1.8 mL in vitro release buffer (100 mM PO4 buffer, 0.025% Tween-20, 0.02% NaN3, 290 mOsm/Kg, pH 6.5) which were placed in a climate chamber thermostated at 37° C. At predetermined time points (2 h, 1 day, 3 days, 7 days, 10 days, 14 days, and then once a week until complete release), vials were collected and centrifuged, whereafter 1.6 mL of buffer was aspirated from the vials and replaced with 1.6 mL of fresh IVR buffer. Aliquots of 800 μL release buffer were collected and stored refrigerated until analysis. OCT concentrations in the aliquoted release samples were determined via reversed phase ultra performance liquid chromatography (“UPLC”) with FLR-detection using a Waters Acquity H-Class UPLC system, equipped with a FLR detector, and a Waters Acquity CSH C18 column (2.1×50 mm, 1.7 μm) maintained at 40° C. Mobile phase A consisted of Water/Acetonitrile/TFA mixed in ratio 90/10/0.1 v/v/v % and Water/Acetonitrile/TFA mixed in ratio 10/90/0.1 v/v/v was used as mobile phase B. The mobile phase composition started at 10% B and increased to 50% B in 2.5 minutes, at a constant flow rate of 0.600 mL/min. At 2.6 minutes the flow was reverted to 10% B and maintained up to a total run time of 3.5 minutes. FLR detection with λ_{excitation} of 280 nm, λₑₘₘᵢₛₛᵢₒₙ of 330 nm was used for quantification of OCT concentration.

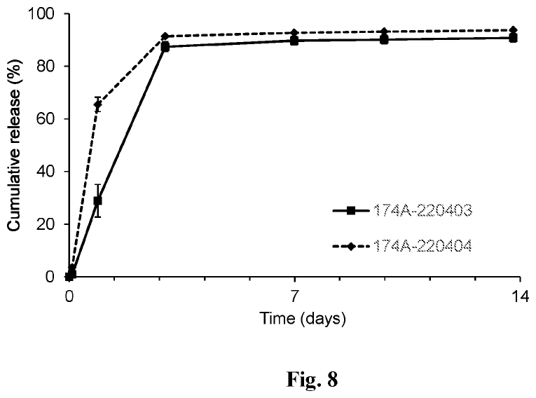

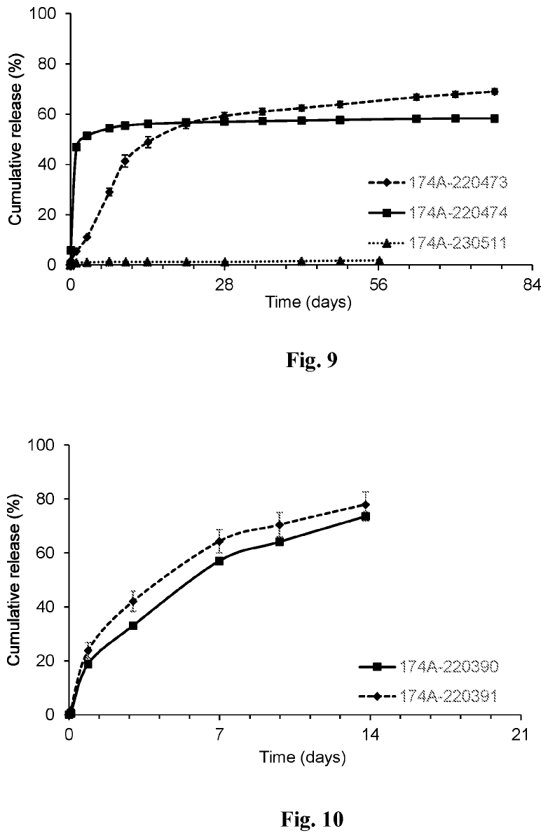

The characteristics of the obtained microspheres are summarized in Table 7 and Table 8. Spherical microparticles with a smooth surface morphology (FIG. 7), an average particle size of 40-50 μm, and narrow particle size distribution (COV 23-27%) were obtained. For all OCT-MSP, irrespective of target OCT loading, high encapsulation efficiency (“EE”) of >90% was obtained. The release of OCT from these OCT-MSP (FIG. 8, FIG. 9) was relatively fast except for the OCT-MSP prepared of 90LP2L25-GLL40 (3/97) which did not show any release up to 8 weeks.

TABLE 7
Characteristics of OCT-MSP with 20 wt. % target OCT
loading prepared of 10LP10L20-GLL40 (15/85) via
W/O/W membrane emulsification with different CP.

| | | | Target | | | OCT | |
|---|---|---|---|---|---|---|---|
| | | | loading | D50 | COV | loading | EE |
| Batch | CP (W₂) | W1/O | (wt. %) | (μm) | (%) | (wt %) | (%) |
| 174A- | 0.4 wt % PVA + 5 wt % | 1:12 | 20 | 51 | 26 | 18.1 | 91 |
| 220403 | NaCl in UPW | | | | | | |
| 174A- | 0.4 wt. % PVA + 5 wt % | 1:12 | 20 | 44 | 26 | 18.1 | 91 |
| 220404 | NaCl in 50 mM TRIS | | | | | | |
| | buffer pH 8.3 | | | | | | |

TABLE 8
Characteristics of OCT-MSP with 10 or 15 wt. % target OCT loading
prepared of different polymers via W/O/W membrane emulsification
with 0.4 wt. % PVA/5 wt. % NaCl solution as CP.

| | | | Target | | | OCT | |
|---|---|---|---|---|---|---|---|
| | | | loading | D50 | COV | loading | EE |
| Batch | Polymer | W1/O | (wt. %) | (μm) | (%) | (wt. %) | (%) |
| 174A- | 10CP6C12- | 1:17 | 15 | 37 | 23 | <LLQ | N.A |
| 220472 | D27 | | | | | | |
| 174A- | 10LP2L20- | 1:17 | 15 | 40 | 25 | 14.0 | 95 |
| 220473 | GLL40 | | | | | | |
| | (3/97) | | | | | | |
| 174A- | 10GL20- | 1:17 | 15 | 37 | 27 | 13.8 | 92 |
| 220474 | L40 | | | | | | |
| 174A- | 10LP2L20- | 1:27 | 10 | 48 | 27 | 9.5 | 93 |
| 220511 | GLL40 | | | | | | |
| | (3/97) | | | | | | |

### Example 4: Octreotide Microsphere Formulations Via O/W Emulsification (1)

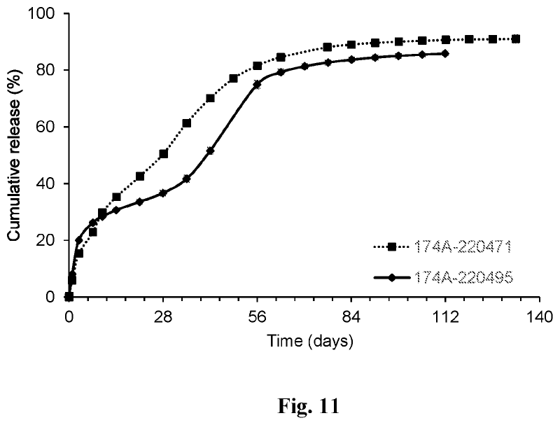

Octreotide-loaded microspheres with a target loading of 20 wt. % were prepared of 10LP10L20-GLL40 (15/85) at a scale of 2 g according to an oil-in-water (“O/W”) membrane emulsification procedure. OA and polymer 10LP10L20-GLL40 (15/58) were dissolved in a 90/10 (w/w) co-solvent mixture of DCM with methanol (MeOH) or acetic acid (AA) to a concentration of 43.3 mg/g OCT and 15 wt. % polymer. The so-obtained DP was filtered over 0.2 μm PTFE filter and subsequently emulsified with CP (aqueous 0.4 wt. % PVA/5 wt. % NaCl solution) by pumping DP at a rate of 1 mL/min via a stainless steel membrane with 20 μm pores into a 2 L stirred vessel into which CP was pumped at a CP/DP ratio of 150 (v/v). Solvent removal and lyophilization of the microspheres were performed as described in example 3. The characteristics of the obtained microspheres are summarized in Table 9. The release of OCT from OCT-MSP is shown in FIG. 10.

TABLE 9
Characteristics of OCT-MSP with 20 wt. % OCT target
loading prepared of 10LP10L20-GLL40 (15/85) by O/W
membrane emulsification using different cosolvents.

| | | Target | | | OCT | |
|---|---|---|---|---|---|---|
| | | loading | D50 | COV | loading | EE |
| Batch# | Co-solvent | (wt. %) | (μm) | (%) | (wt. %) | (%) |
| 174A-220390 | MeOH | 20.2 | 161 | 58 | 19.5 | 94 |
| 174A-220391 | Acetic acid | 19.8 | 163 | 66 | 18.2 | 90 |

### Example 5: Octreotide Microsphere Formulations Via O/W Emulsification (2)

Octreotide-loaded microspheres with a target loading of 15 wt. % OCT were prepared of 10LP2L20-GLL40 (3/97) and 10CP6C12-D27 at a scale of 2 g according to the O/W membrane emulsification procedure described in example 4. OA and polymers were dissolved in a 90/10 (w/w) co-solvent mixture of DCM and MeOH to a concentration of 20.2 mg/g OCT and 10 wt. % polymer or 30.3 mg/g OCT and 15 wt. % polymer. The so-obtained DP was filtered over 0.2 μm PTFE filter and subsequently emulsified with CP (aqueous 0.4 wt. % PVA/5 wt. % NaCl solution) by pumping DP at a controlled flow rate via a membrane with 40 μm pores into a 2 L stirred vessel into which CP was pumped at a CP/DP ratio of 150 v/v. Solvent removal and lyophilization of the microspheres was performed as described in example 2. The characteristics of the obtained microspheres are summarized in Table 10. The release of OCT from OCT-MSP is shown in FIG. 11.

TABLE 10
Characteristics of OCT-MSP with 15 wt. % OCT target
loading prepared of by O/W emulsification using
different polymers and polymer concentrations.

| | | Pol. | OCT | | | OCT | |
|---|---|---|---|---|---|---|---|
| | | conc. | in DP | D50 | COV | loading | EE |
| Batch# | Polymer | (wt. %) | (mg/g) | (μm) | (%) | (wt. %) | (%) |
| 174A- | 10CP6C12- | 10 | 20.2 | 55 | 30 | <LLQ | N.A |
| 220470 | D27 | | | | | | |
| 174A- | 10LP2L20- | 10 | 20.3 | 50 | 31 | 14.4 | 99 |
| 220471 | GLL40 | | | | | | |
| | (3/97) | | | | | | |
| 174A- | 10LP2L20- | 15 | 30.3 | 64 | 40 | 14.5 | 97 |
| 220495 | GLL40 | | | | | | |
| | (3/97) | | | | | | |

### Example 6: Octreotide Microsphere Formulations Via O/W Emulsification (3)

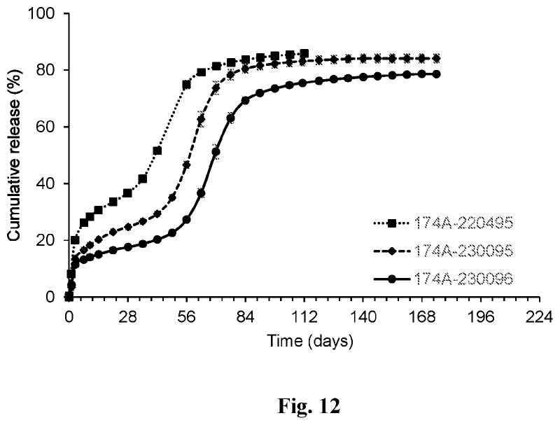

Octreotide-loaded microspheres with a target loading of 15 wt. % were prepared of 10LP2L20-GLL40 (3/97) at a scale of 2-5 g according to the O/W membrane emulsification procedure described in example 5. OA and 10LP2L20-GLL40 (3/97) were dissolved in a 90/10 (w/w) co-solvent mixture of DCM and MeOH to a concentration of 30.3 mg/g OCT and 15 wt. % polymer. The so-obtained DP was filtered over 0.2 μm PTFE filter and subsequently emulsified with CP (aqueous 0.4 wt. % PVA/5 wt. % NaCl solution) by pumping DP at a controlled flow rate via a membrane with 40 μm pores into a stirred vessel into which CP was pumped at a CP/DP ratio of 150 v/v. Solvent removal was performed according to the general procedures described in example 2 whereby the air flow, volume of the vessel and extraction temperature were varied (Table 11). Lyophilization of the microspheres was performed as described in example 2. The characteristics of the obtained microspheres are summarized in Table 12. The release of OCT from OCT-MSP is shown in FIG. 12.

TABLE 11
Batch size and extraction conditions used for the preparation
of 10LP2L20-GLL40 (3/97)-based OCT-MSP with 15 wt. % OCT
target loading using the O/W emulsification procedure.

| | | Pol | | OCT | Air | Volume | Bottle | Extraction | |
|---|---|---|---|---|---|---|---|---|---|
| | Scale | conc. | MeOH | target | flow | in vessel | size | time (h) | |
| Batch# | (g) | (wt. %) | % | loading | (L/min) | (mL) | (L) | RT | 45° C. |
| 174A- | 2.0 | 14.9 | 11.3 | 14.9 | 5 | 1000 | 4 | 3 | — |
| 220495 | | | | | | | | | |
| 174A- | 4.5 | 15.0 | 10.0 | 16.1 | 10 | 1500 | 3 | 3 | — |
| 230095 | | | | | | | | | |
| 174A- | 4 | 15.0 | 10.0 | 16.1 | 10 | 3000 | 5 | 1 | 2 |
| 230096 | | | | | | | | | |

TABLE 12
Characteristics of 10LP2L20-GLL40 (3/97)-based OCT-MSP with 15
wt. % OCT target loading prepared by O/W membrane emulsification
using different batch sizes and extraction conditions.

| | | | | | | OCT | |
|---|---|---|---|---|---|---|---|
| | Scale | Extraction | D50 | COV | DCM | loading | EE |
| Batch# | (g) | temperature | (μm) | (%) | (ppm) | (wt. %) | (%) |
| 174A- | 2.0 | RT | 64 | 40 | >5500 | 14.5 | 97 |
| 220495 | | | | | | | |
| 174A- | 4.5 | RT | 53 | 26 | >5500 | 14.3 | 89 |
| 230095 | | | | | | | |
| 174A- | 4 | RT + 45° C. | 54 | 27 | 218.9 | 14.6 | 91 |
| 230096 | | | | | | | |

### Example 7: Octreotide Microsphere Formulations Via O/W Emulsification (4)

Octreotide-loaded microspheres with a target loading of 10 and 15 wt. % were prepared of 10LP2L20-GLL40 (3/97) at a scale of 5 g according to the O/W membrane emulsification procedure described in example 6. OA and polymers were dissolved in a 90/10 (w/w) co-solvent mixture of DCM with MeOH to a concentration of 20.2 mg/g or 30.3 mg/g OCT and 15 wt. % polymer. The so-obtained DP was filtered over 0.2 μm PTFE filter and subsequently emulsified with CP (aqueous 0.4 wt. % PVA/5 wt. % NaCl solution) by pumping DP at a controlled flow rate via a membrane with 40 μm pores into a 5 L stirred vessel into which CP was pumped at a CP/DP ratio of 150 v/v. Solvent removal was performed by stirring the suspension under an air flow of 10 L/min for 1 hour at RT followed by 2 hours at 45° C. Lyophilization of the microspheres was performed as described in example 3.

Each formulation (10 wt. % or 15 wt. % target OCT loading) was produced in triplicate and analysed for morphology (SEM), particle size distribution, OCT loading, residual solvent content, and burst release (percentage released at 2 hours), whereafter the individual batches were blended to obtain a master batch of ~15 g for each formulation. Each master batch was additionally vacuum dried at 45° for 18 hours.

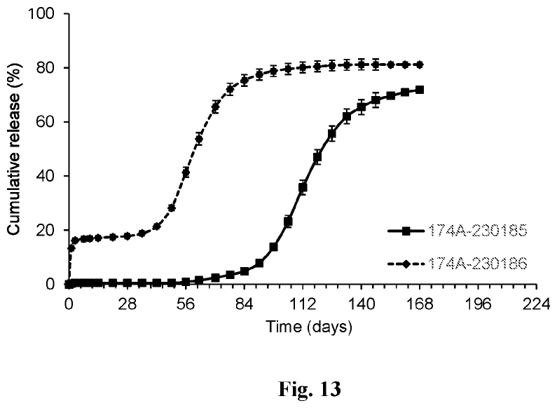

The characteristics of the individual OCT-MSP batches are summarized in Table 13 and the characteristics of the two master batches of OCT-MSP are summarized in Table 14. The release of OCT from OCT-MSP is shown in FIG. 13. OCT release from OCT-MSP containing 15 wt OCT exhibited a significant burst release (13.4%) in one day whereafter sigmoidal release kinetics with a release duration of around 3-4 months were obtained. OCT-MSP with an OCT loading of 10 wt. % released their payload significantly different. An exceptional low burst release of 0.3 was followed by a lag time of round 60 days, whereafter OCT was gradually released according to sigmoidal profile. Based on extrapolation of the cumulative release data the effective duration of release (from onset to completion of release) is predicted to be 4-5 months.

TABLE 13
Characteristics of individual OCT-MSP batches prepared. All batches
were prepared using 10LP2L20-GLL40 (3/97) polymer and O/W process.

| | | | | | | | | | | | Burst |
|---|---|---|---|---|---|---|---|---|---|---|---|
| | Target | Pol. | | OCT | | | | | OCT | | release |
| | loading | Conc. | MeOH | in DP | D50 | COV | MeOH | DCM | loading | EE | at 1 day |
| Batch# | (wt. %) | (wt. %) | (wt. %) | (mg/g) | (μm) | (%) | (ppm) | (ppm) | (wt. %) | (%) | (%) |
| 174A- | 10 | 15 | 10 | 20.2 | 62.3 | 55.6 | <1030 | 2297 | 10.57 | 105.5 | 0.2 |
| 230164 | | | | | | | | | | | |
| 174A- | 10 | 15 | 10 | 20.2 | 49.6 | 34.0 | <1030 | 2740 | 10.66 | 106.4 | 0.3 |
| 230165 | | | | | | | | | | | |
| 174A- | 10 | 15 | 10 | 20.2 | 51.9 | 33.3 | <1030 | >5500 | 11.07 | 110.1 | 0.3 |
| 230166 | | | | | | | | | | | |
| 174A- | 15 | 15 | 10 | 30.3 | 53.6 | 28.6 | <1030 | 1256 | 16.38 | 109.7 | 10.1 |
| 230168 | | | | | | | | | | | |
| 174A- | 15 | 15 | 10 | 30.3 | 53.0 | 31.9 | <1030 | 270 | 16.71 | 109.3 | 11.3 |
| 230169 | | | | | | | | | | | |
| 174A- | 15 | 15 | 10 | 30.3 | 58.5 | 34.4 | <1030 | 761 | 16.82 | 110.2 | 13.2 |
| 230170 | | | | | | | | | | | |

TABLE 14
Characteristics of OCT-MSP master batches with 10 and 15 wt. % OCT target loading
prepared of 10LP2L20-GLL40 (3/97) polymer via the O/W emulsification process.

| | | 174A-230185 | | 174A-230186 | |
|---|---|---|---|---|---|
| Parameter | Test method | (10 wt. % OCT) | | (15 wt. % OCT) | |
| Polymer grade | N.A | 10LP2L20-GLL40 (G/LL 3/97) | | 10LP2L20-GLL40 (G/LL 3/97) | |
| Appearance | Visual inspection | White to off-white powder | | White to off-white powder | |
| Microscopic | SEM | Spherical particles | | Spherical particles | |
| appearance | | | | | |
| Average particle | Laser diffraction | 51 | μm | 54 | μm |
| size (Dᵥ50) | | | | | |
| Coefficient of | Laser diffraction | 29% | | 39% | |
| variance (COV) | | | | | |
| OCT loading in | BCA assay | 10.0 | wt. % | 15.2 | wt. % |
| microsphere | | | | | |
| Encapsulation | BCA assay | 107% | | 104% | |
| efficiency | | | | | |
| Residual | GC | MeOH: <1030 ppm | | MeOH: <1030 ppm | |
| solvent(s) | | DCM: 683 ppm | | DCM: 180 ppm | |
| content | | | | | |
| Burst release | Dissolution test | 0.3% | | 13.4% | |
| at 1 day | | | | | |
| Syringeability | Injectability test | 50 wt./vol. % suspension conc. | | 33 wt./vol. % suspension conc. | |
| (18 gauge | | injectable; max force 4.9N | | injectable; max force 7.3N | |
| needle) | | | | | |
| Endotoxin | LAL test | <0.01 | EU/mg | <0.98 | EU/mg |
| content | | | | | |

### Example 8: In Vivo PK/PD Study

An in vivo study was performed in beagle dogs to study the pharmacokinetics and pharmacodynamics of the octreotide loaded microspheres. OCT-MSPs prepared as described in example 7 containing 10 wt. % OCT (174A-230185) and 15 wt. % OCT (174A-230186) were used. 800-1200 mg of lyophilized octreotide microspheres were reconstituted in 1.7 and 1.3 mL of diluent (WFI containing 0.6 wt. % sodium carboxymethyl cellulose) respectively as to obtain a microsphere suspension concentration of 33-50 wt./vol % and 2 mL of the suspension was administered to beagle dogs via subcutaneous injection.

### Example 9: Octreotide Microsphere Formulations Via O/W Emulsification (Scale-Up)

Octreotide-loaded microspheres with a target loading of 10 wt. % were prepared of 10LP2L20-GLL40 (3/97) at a scale of 15 g by O/W membrane emulsification using an advanced crossflow assembly (AXF™, MicroPore) (“AXF”). OA and polymer were dissolved in a 90/10 (w/w) co-solvent mixture of DCM and MeOH to a concentration of 20.2 mg/g OCT and 15 wt. % polymer. The so-obtained DP was filtered over 0.2 μm PTFE filter and subsequently emulsified with CP (aqueous 0.4 wt. % PVA/5 wt. % NaCl solution) by pumping DP and CP at controlled flow rates and final CP/DP ratio of 150 v/v via the AXF assembly equipped with a 20 μm pore size tubular membrane. The formed suspension was collected in a 10 L stirred vessel. Solvent removal was performed by hollow fiber diafiltration for one hour with a volume exchange rate of 30 times per hour. Lyophilization of the microspheres was performed according to the procedures described in example 2.

Spherical microparticles with a smooth surface morphology and slightly larger particle size (D50 63.5 μm, COV 42.6%) as obtained in example 7 were obtained. OCT content of the microparticles was 10.5 wt. % (EE 104.6%).

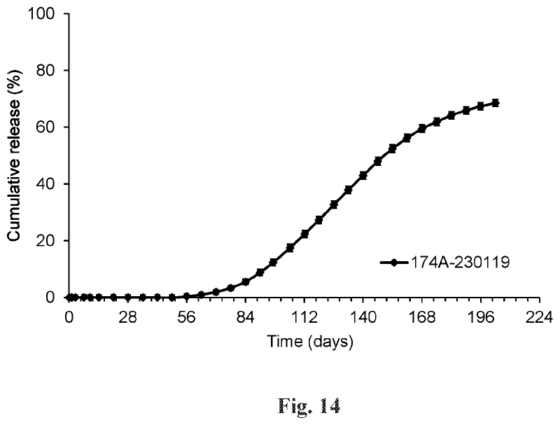

Cumulative in vitro release kinetics of OCT as shown in FIG. 14 were similar to the release kinetics obtained for OCT-MSP batch 174A-230185 of example 7. After a lag time of around 60 days, OCT was gradually released. Based on extrapolation of the cumulative release data the effective duration of release (from onset to completion of release) is predicted to be ~5 months.

1.-74. (canceled)

75. An extended release octreotide dosage form comprising:
(a) octreotide, or a pharmaceutically acceptable salt thereof; and
(b) a biodegradable polymer microsphere;
wherein the octreotide, or the pharmaceutically acceptable salt thereof, is present in the biodegradable polymer microsphere,
wherein the biodegradable polymer microsphere comprises about 5 wt % to 99 wt % of a biodegradable polymer,
wherein the biodegradable polymer is a multiblock copolymer comprising at least one hydrolysable prepolymer (A) segment and at least one hydrolysable prepolymer (B) segment, wherein the segments are linked by a multifunctional chain extender,
wherein the prepolymer (B) segment comprises poly(lactide-co-glycolide) with a glycolide/lactide monomer molar ratio of about 1:100 to 100:1.

76. The dosage form of claim 75, wherein the prepolymer (A) segment comprises one or more linkages selected from the group consisting of ester linkages, carbonate linkages, anhydride linkages, ether linkages, and combinations thereof.

77. The dosage form of claim 75, wherein the prepolymer (A) segment comprises products of a reaction of at least one cyclic monomer with at least one noncyclic initiator selected from the group consisting of diols, dicarboxylic acids, and hydroxycarboxylic acids.

78. The dosage form of claim 77, wherein the at least one cyclic monomer is selected from the group consisting of glycolide, lactide (D and/or L), ε-caprolactone, δ-valerolactone, trimethylene carbonate, 1,4-dioxan-2-one (para-dioxanone), 1,5-dioxan-2-one, and a cyclic anhydride.

79. The dosage form of claim 77, wherein the noncyclic initiator is selected from the group consisting of succinic acid, glutaric acid, adipic acid, sebacic acid, lactic acid, glycolic acid, ethylene glycol, diethylene glycol, 1,4-butanediol, and 1,6-hexanediol.

80. The dosage form of claim 75, wherein the prepolymer (A) segment comprises reaction products of glycolide, lactide (D and/or L), ε-caprolactone, and/or δ-valerolactone.

81. The dosage form of claim 75, wherein the amount of hydrolysable prepolymer (A) segment in the biodegradable multiblock copolymer is about 5% to about 90% based on total weight of the biodegradable multiblock copolymer.

82. The dosage form of claim 75, wherein the prepolymer (A) segment has a Mₙ of about 500 g/mol or more.

83. The dosage form of claim 75, wherein the at least one hydrolysable prepolymer (A) segment comprises poly(ethylene glycol) (PEG) with a Mₙ of about 150 to about 5000 g/mol.

84. The dosage form of claim 75, wherein the at least one hydrolysable prepolymer (A) segment comprises reaction products of ester forming monomers selected from glycolide, lactide (D and/or L), ε-caprolactone, and δ-valerolactone.

85. The dosage form of claim 75, wherein the at least one hydrolysable prepolymer (A) segment is poly(D,L-lactide)-co-poly(ethylene glycol)-co-poly(D,L-lactide).

86. The dosage form of claim 75, wherein the prepolymer (B) segment comprises a polymer derived from hydroxyalkanoate, glycolide, lactide (D and/or L), ε-caprolactone, δ-valerolactone, trimethylene carbonate, 1,4-dioxan-2-one or combinations thereof.

87. The dosage form of claim 75, wherein the at least one hydrolysable prepolymer (B) segment comprises poly(L-lactide-co-glycolide).

88. The dosage form of claim 75, wherein the prepolymer (B) segment comprises poly(lactide-co-glycolide) with a glycolide/lactide monomer molar ratio of about 1:20 to 20:1.

89. The dosage form of claim 75, wherein the prepolymer (B) segment comprises poly(lactide-co-glycolide) with a glycolide/lactide monomer molar ratio of about 1:5 to 5:1.

90. The dosage form of claim 75, wherein the prepolymer (B) segment comprises poly(L-lactide-co-glycolide) with a glycolide/L-lactide monomer molar ratio of about 0.2 or more.

91. The dosage form of claim 75, wherein the prepolymer (B) segment comprises poly(L-lactide-co-glycolide) with a glycolide/L-lactide monomer molar ratio of about 0.4 or more.

92. The dosage form of claim 75, wherein the prepolymer (B) segment comprises poly(L-lactide-co-glycolide) with a glycolide/L-lactide monomer molar ratio of about 0.6 or more.

93. The dosage form of claim 75, wherein the prepolymer (B) segment further comprises poly(p-dioxanone) with a ML of about 1000 g/mol or more.

94. The dosage form of claim 75, wherein the multiblock copolymer comprises from about 10 wt % to about 99 wt % of the prepolymer (B) segment relative to the total weight of the multiblock copolymer.

95. The dosage form of claim 75, wherein the multifunctional chain extender is a difunctional aliphatic chain extender.

96. The dosage form of claim 75, wherein the multifunctional chain extender is a diisocyanate.

97. The dosage form of claim 75, wherein the segments are randomly distributed over the chain of the biodegradable multiblock copolymer.

98. The dosage form of claim 75, wherein the biodegradable polymer microsphere has a swelling ratio under physiological conditions of about 1 to about 5.

99. The dosage form of claim 75, wherein the biodegradable multiblock copolymer comprises a [poly(D,L-lactide)-co-poly(ethylene glycol)-co-poly(D,L-lactide)]-b-[poly(L-lactide-co-glycolide)] multiblock copolymer.

100. The dosage form of claim 75, further comprising an aqueous diluent.

101. The dosage form of claim 100, wherein the diluent comprises carboxymethyl cellulose aqueous solution.

102. The dosage form of claim 100, wherein the microspheres are suspended in the aqueous diluent.

103. The dosage form of claim 75, comprising about 5 wt % to about 60 wt % of the biodegradable polymer microsphere.

104. The dosage form of claim 75, wherein the microsphere comprises about 1 wt % to about 50 wt % of the octreotide, or the pharmaceutically acceptable salt thereof.

105. The dosage form of claim 75, wherein the microsphere comprises about 5 wt % to about 30 wt % of the octreotide, or the pharmaceutically acceptable salt thereof.

106. The dosage form of claim 75, wherein the pharmaceutically acceptable salt of octreotide comprises one or more selected from the group consisting of an acetate salt of octreotide, a trifluoroacetate salt of octreotide, and a hydrochloride salt of octreotide.

107. The dosage form of claim 75, wherein the biodegradable polymer microsphere comprises about 10 wt % to about 99 wt % of a biodegradable polymer.

108. The dosage form of claim 75, further comprising a surfactant.

109. The dosage form of claim 108, comprising about 0.01 wt % to about 20 wt % of the surfactant.

110. The dosage form of claim 108, wherein the surfactant is selected from the group consisting of polysorbates, poloxamers, bile salts, glyceryl monostearate, sodium lauryl sulfate, sorbitan monooleate, polyoxyethylene sorbitan monooleate, copolymers of ethylene oxide and propylene oxide, d-α-tocopheryl polyethylene glycol succinate (Vitamin E TPGS), or a combination thereof.

111. The dosage form of claim 75, wherein the first 20 days after being administered to a mammal, the biodegradable polymer microsphere releases from about 5 wt % to about 50 wt % of the octreotide, or the pharmaceutically acceptable salt thereof, present in the biodegradable polymer microsphere.

112. The dosage form of claim 75, wherein the first 60 days after being administered to a mammal, the biodegradable polymer microsphere releases from about 30 wt % to about 90 wt % of the octreotide, or the pharmaceutically acceptable salt thereof, present in the biodegradable polymer microsphere.

113. The dosage form of claim 75, wherein the first 20 days after being administered to a mammal, the biodegradable polymer microsphere releases at most about 10 wt % of the octreotide, or the pharmaceutically acceptable salt thereof, present in the biodegradable polymer microsphere.

114. The dosage form of claim 75, wherein the first 60 days after being administered to a mammal, the biodegradable polymer microsphere releases at most about 50 wt % of the octreotide, or the pharmaceutically acceptable salt thereof, present in the biodegradable polymer microsphere.

115. Kit of formulation comprising:
(a) a first container comprising a microsphere, wherein the microsphere comprises octreotide, or a pharmaceutically acceptable salt thereof and a biodegradable polymer,
wherein the biodegradable polymer microsphere comprises about 10 wt % to 99 wt % of a biodegradable polymer,
wherein the biodegradable polymer is a multiblock copolymer comprising at least one hydrolysable prepolymer (A) segment and at least one hydrolysable prepolymer (B) segment, wherein the segments are linked by a multifunctional chain extender,
wherein the prepolymer (B) segment comprises poly(lactide-co-glycolide) with a glycolide/lactide monomer molar ratio of about 1:100 to 100:1; and
(b) a second container comprising a pharmaceutically acceptable diluent.
